(19) United States
(12) Patent Application Publication
(10) Pub. No.: US 2015/0197492 A1
(43) Pub. Date: Jul. 16, 2015

(54) NOVEL INDENO[1,2-c]QUINOLIN-11-ONE DERIVATIVES, PREPARATION METHOD AND APPLICATION THEREOF

(71) Applicant: National Defense Medical Center, Taipei (TW)

(72) Inventors: Hsu-Shan Huang, Taipei City (TW); Tsung-Chih Chen, Nantou County (TW); Shiang-Jiun Chen, Taipei City (TW); Chun-Liang Chen, Taipei City (TW); Chia-Chung Lee, Taipei City (TW)

(73) Assignee: National Defense Medical Center, Taipei (TW)

(21) Appl. No.: 14/153,333

(22) Filed: Jan. 13, 2014

(51) Int. Cl. C07D 221/18; C07D 401/14; C07D 491/113; C07D 401/04; C07D 401/12

(52) U.S. Cl. CPC C07D 221/18; C07D 401/04; C07D 401/12; C07D 491/113; C07D 401/14

(57) ABSTRACT

The present invention provides a novel series of indeno[1,2-c]quinolin-11-one derivatives and further provides their preparation methods as well as applications. Said applications includes utilizing such derivatives as pharmaceutical compositions for treating cancers; moreover, said applications includes the capability possessed by such derivatives to effectively inhibit cancer cell growth as well as the activity of Type I topoisomerases and can be further applied for cancer treatment.

### BACKGROUND OF THE INVENTION

1. Field of the Invention

This invention relates to the development of anticancer drugs, in particular, featuring a series of novel indeno[1,2-c]quinolin-11-one derivatives, preparation methods and applications as well as the evaluation of the anticancer activity of the inventive drugs.

2. Description of the Prior Art

Cancers arise from the abnormal proliferation of DNA. Therefore, selectively destroying the DNA of cancer cells without damaging the DNA of normal cells is highly desired. However, it is difficult to differentiate the DNAs between normal cells and cancer cells. Consequently, specific ‘targeted therapy’ was developed following the identification of the differences between normal cells and cancer cells, and when combined with other chemotherapies or radiation therapies, targeted therapy can significantly reduce the side effects and provide better treatment outcomes. Thus, targeted therapy currently is a popular field in studying cancer treatments. Because topoisomerases have been found to play an indispensible role in DNA replication, they have become the object of targeted therapy for anticancer treatments. The anticancer drug camptothecin discovered by M. E. Wall and M. C. Wani in 1966 through systematic screening of natural substances is an inhibitor for type I topoisomerases.

Unfortunately, camptothecin has numerous disadvantages and thus cannot be used for clinical treatment. For example, the lactone ring can be easily hydrolyzed to hydroxycarboxylate in vivo at the normal pH and then binds to serum albumin and lose its effect of inhibiting the function of type I topoisomerases. In addition, the structure of the tricomplex of camptothecin-Top I-DNA is not stable because the complex is not maintained by covalent bonds and water solubility of camptothecin is poor which causes lower bioavailability. The p-glycoprotein (MDR1, ABCB1) efflux transporter proteins in the cell membrane transport the drugs out of the cells and more important is that some tumor cells have slowly developed resistance and adverse drug side effects against camptothecin. As a result, a number of water-soluble semi-synthetic drugs were developed even after commercialization of camptothecin such as Topotecan (HYCAMTIN®) which is used for treating ovarian cancer and Irinotecan (CAMPTO®) which is used for treating colon cancer and both have issues when used for clinical treatment.

Hence, based on the importance of topoisomerase inhibitors in development of anticancer drugs, the inventor of this application developed a series of novel indeno[1,2-c]quinolin-11-one derivatives and disclosed the preparation methods as well as relevant applications herein after a number of innovative improvements.

### SUMMARY OF THE INVENTION

In one aspect, present invention provides a formula I compound:

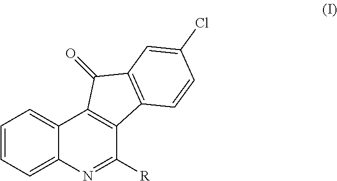

wherein the R group is selected from the groups consisting of: i) haloformyl, amino, hydroxy and thiol groups, ii) linear alkyl chains of N(CH₂)ₙH, alkyl groups with substituted side chains and alkyl side chains with a substituted amino group, NH(CH₂)ₙN(CH₂)ₙ, O(CH₂)ₙ and S(CH₂)ₙOH, wherein 1≦n≦10, iii) nitrogen-containing cycloalkyl groups and heterocyclic compounds of C₃₋₁₂ which contain 1 to 3 heteroatoms selected from O, S and N, wherein the ortho-, para- and meta-position can be further selected independently from one of the following functional groups comprising (CH₂)ₙ alkyl groups, (CH₂)ₙC₃₋₁₂ cycloalkyl groups, (CH₂)ₙC₃₋₁₂ nitrogen-containing cycloalkyl groups, (CH₂)ₙ benzene rings and (CH₂)ₙCOC₃₋₁₂ nitrogen-containing cycloalkyl groups, wherein 0≦n≦10, wherein the nitrogen-containing cycloalkyl groups or the benzene rings can be further substituted by one or more substitution groups selected from the following groups comprising alkyl groups containing C₁₋₁₂, amino groups, nitro groups, hydroxyl groups, cyano groups, halogen groups, un-substituted or halogen group substituted C₁₋₅ alkyl groups, un-substituted or halogen group substituted alkoxy groups, and their pharmaceutically acceptable salts, stereoisomers and enantimoers.

According to the invention, the functional groups of ii) for substitution are selected from the groups consisting of methylamino group, dimethylamino group, 2-(diethylamino)ethyl-amine and 2-hydroxyethyl ethyl sulfide.

According to the invention, the functional groups of iii) is selected from the groups consisting of pyrrolidin-1-yl, piperidin-1-yl, 4-methyl-piperazin-1-yl, azepan-1-yl, morpholino, thiomorpholino, piperazin-1-, 2-methyl-piperazin-1-yl, 4-methyl-piperazin-1-yl, 4-ethyl-piperazin-1-yl, 4-cyclopentyl-piperazin-1-yl, 4-(piperidin-1-yl) piperidin-1-yl, 4-phenyl-piperazin-1-yl, 4-benzyl-piperazin-1-yl, 4-(2-fluorophenyl)piperazine-1-yl, 4-(2-methoxyphenyl)piperazin-1-yl, 4-(3-methoxyphenyl)piperazin-1-yl, 4-(1-methyl-piperidin-4-yl)piperazin-1-yl, 4-(1,4-dioxo-8-aza-spiro[4,5]dec-8-yl, 4-((piperazin-1-yl) (piperidin-1-yl) methanone), 4-(3-(piperidin-4-yl) propyl) piperidin-1-yl, hydroxy, and methoxy.

According to the invention, the compound is selected from the groups consisting of
9-Chloro-6-(methylamino)-11H-indeno[1,2-c]quinolin-11-one,
9-chloro-6-(dimethylamino)-11H-indeno[1,2-c]quinolin-11-one,
9-chloro-6-(pyrrolidin-1-yl)-11H-indeno[1,2-c]quinolin-11-one,
9-chloro-6-(piperidin-1-yl)-11H-indeno[1,2-c]quinolin-11-one,
9-chloro-6-(4-methyl-piperazin-1-yl)-11H-indeno[1,2-c]quinolin-11-one,
6-(azepan-1-yl)-9-chloro-11H-indeno[1,2-c]quinolin-11-one,
9-chloro-6-morpholino-11H-indeno[1,2-c]quinolin-11-one,
9-chloro-6-thiomorpholino-11H-indeno[1,2-c]quinolin-11-one,
9-chloro-6-(piperazin-1-yl)-11H-indeno[1,2-c]quinolin-11-one,
9-chloro-6-(2-methyl-piperazin-1-yl)-11H-indeno[1,2-c]quinolin-11-one,
9-chloro-6-(4-methyl-piperazin-1-yl)-11H-indeno[1,2-c]quinolin-11-one,
9-chloro-6-(4-ethyl-piperazin-1-yl)-11H-indeno[1,2-c]quinolin-11-one,
9-chloro-6-(4-cyclopentyl-piperazin-1-yl)-11H-indeno[1,2-c]quinolin-11-one,
9-chloro-6-(4-(piperidin-1-yl) piperidin-1-yl)-11H-indeno[1,2-c]quinolin-11-one,
9-chloro-6-(4-phenyl-piperazin-1-yl)-11H-indeno[1,2-c]quinolin-11-one,
6-(4-benzyl-piperazin-1-yl)-11H-indeno[1,2-c]quinolin-11-one,
9-chloro-6-(4-(2-fluorophenyl)piperazin-1-yl)-11H-indeno[1,2-c]quinolin-11-one,
9-chloro-6-(4-(2-methoxyphenyl)piperazin-1-yl)-11H-indeno[1,2-c]quinolin-11-one,
9-chloro-6-(4-(3-methoxyphenyl)piperazin-1-yl)-11H-indeno[1,2-c]quinolin-11-one,
9-chloro-6-(4-(1-methyl-piperidin-4-yl)piperazin-1-yl)-11H-indeno[1,2-c]quinolin-11-one,
9-chloro-6-(4-(1,4-dioxo-8-aza-spiro[4,5]dec-8-yl)-11H-indeno[1,2-c]quinolin-11-one,
9-chloro-6-(4-((piperazin-1-yl) (piperidin-1-yl)methanone))-11H-indeno[1,2-c]quinolin-11-one, and
9-chloro-6-(4-(3-(piperidin-4-yl) propyl) piperidin-1-yl)-11H-indeno[1,2-c]quinolin-11-one,
6-(2-Hydroxyethylthio)-9-chloro-11H-indeno[1,2-c]quinolin-11-one,
6-hydroxy-9-chloro-11H-indeno[1,2-c]quinolin-11-one,
6-methoxy-9-chloro-11H-indeno[1,2-c]quinolin-11-one, and their salts.

In another aspect, the invention provides a pharmaceutical composition comprising of the effective dose of the abovementioned compound and at least one of the pharmaceutically acceptable vehicles, diluents or excipients.

In the other aspect, present invention provides an application of the abovementioned compound which can be used to produce pharmaceutical products capable of inhibiting the activity of type I topoisomerases.

In still another aspect, the invention provides an application of the abovementioned compound which can be used to produce pharmaceutical products for cancer treatment.

In one aspect, the invention provides a method for inhibiting type I topoisomerase activity including administration of an effective dose of the abovementioned compound.

According to the invention, the cancers are selected from the following list: leukemia, non-small cell lung cancer, colorectal cancer, CNS cancer, melanoma, ovarian cancer, renal cancer, prostate cancer and breast cancer. In another aspect, present invention provides a method used for producing the compound disclosed in claim 1, and the method comprising (1) Mix isatin, 2-(4-chlorophenyl) acetic acid and sodium acetate at 200° C. for 3 hours, allow cooling before adding acetic acid and collect the precipitate following extraction and filtration. Wash the precipitate with acetic acid, water and n-hexane to give an intermediate product and add the intermediate product to phosphoryl trichloride and reflux at 150° C. for 48 hours. Upon completion, allow cooling to room temperature followed by addition of 0° C. ice water. Collect the resulting precipitate after extraction and filtration and place in 10% aqueous sodium bicarbonate solution for 1 hour with vigorous stirring. The crude product was recrystallized from dichloromethane after washing with water to give 6,9-dichloro-11H-indeno[1,2-c]quinoline-11-one (TC-XCl-1), (2) Dissolve 6,9-dichloro-11H-indeno[1,2-c]quinoline-11-one obtained from step 1 in N,N-dimethylformamide and add methylamine or N1,N1-diethylethane-1,2-diamine followed by addition of N,N-diisopropylethylamine to catalyze the reaction at 150° C. for 4 hours. The resulting mixture was poured into ice water and incubated for 10 to 20 minutes to give the precipitate which was then recrystallized by ethanol to produce the compounds 9-chloro-6-(dimethylamino)-11H-indeno[1,2-c]quinolin-11-one (SJ-1) and 6-(2-(diethylamino)ethylamine yl)-11H-indeno[1,2-c]quinolin-11-one (SJ-3), (3) Dissolve the 6,9-dichloro-11H-indeno[1,2-c]quinoline-11-one obtained from step (1) in dimethyl formamide, add secondary amines followed by addition of pyridine to catalyze the reaction at 150° C. for 4 hours. The mixture resulted at the end of reaction was poured into ice water and incubated for 10 to 20 minutes to give the precipitate which was then recrystallized by ethanol to produce the compounds which are selected from the following groups consisting of 9-chloro-6-(dimethylamino)-11H-indeno[1,2-c]quinolin-11-one,
9-chloro-6-(pyrrolidin-1-yl)-11H-indeno[1,2-c]quinolin-11-one,
9-chloro-6-(piperidin-1-yl)-11H-indeno[1,2-c]quinolin-11-one,
9-chloro-6-(4-methyl-piperazin-1-yl)-11H-indeno[1,2-c]quinolin-11-one,
6-(azepan-1-yl)-9-chloro-11H-indeno[1,2-c]quinolin-11-one,
9-chloro-6-morpholino-11H-indeno[1,2-c]quinolin-11-one,
9-chloro-6-thiomorpholino-11H-indeno[1,2-c]quinolin-11-one,
9-chloro-6-(piperazin-1-yl)-11H-indeno[1,2-c]quinolin-11-one,
9-chloro-6-(2-methyl-piperazin-1-yl)-11H-indeno[1,2-c]quinolin-11-one,
9-chloro-6-(4-methyl-piperazin-1-yl)-11H-indeno[1,2-c]quinolin-11-one,
9-chloro-6-(4-ethyl-piperazin-1-yl)-11H-indeno[1,2-c]quinolin-11-one,
9-chloro-6-(4-cyclopentyl-piperazin-1-yl)-11H-indeno[1,2-c]quinolin-11-one,
9-chloro-6-(4-(piperidin-1-yl) piperidin-1-yl)-11H-indeno[1,2-c]quinolin-11-one,
9-chloro-6-(4-phenyl-piperazin-1-yl)-11H-indeno[1,2-c]quinolin-11-one,
6-(4-benzyl-piperazin-1-yl)-11H-indeno[1,2-c]quinolin-11-one,
9-chloro-6-(4-(2-fluorophenyl)piperazin-1-yl)-11H-indeno[1,2-c]quinolin-11-one,
9-chloro-6-(4-(2-methoxyphenyl)piperazin-1-yl)-11H-indeno[1,2-c]quinolin-11-one,
9-chloro-6-(4-(3-methoxyphenyl)piperazin-1-yl)-11H-indeno[1,2-c]quinolin-11-one,
9-chloro-6-(4-(1-methyl-piperidin-4-yl)piperazin-1-yl)-11H-indeno[1,2-c]quinolin-11-one,
9-chloro-6-(4-(1,4-dioxo-8-aza-spiro[4,5]dec-8-yl)-11H-indeno[1,2-c]quinolin-11-one,
9-chloro-6-(4-((piperazin-1-yl) (piperidin-1-yl)methanone))-11H-indeno[1,2-c]quinolin-11-one, and
9-chloro-6-(4-(3-(piperidin-4-yl) propyl) piperidin-1-yl)-11H-indeno[1,2-c]quinolin-11-one; (4) Dissolve 6,9-dichloro-11H-indeno[1,2-c]quinoline-11-one obtained from step 1 in dimethyl formamide and then add 2-mercaptoethanol followed by catalytic reaction of potassium carbonate at 150° C. for 4 hours. At the end of reaction, pour the mixture into ice water and incubate for 10 to 20 minutes. Finally, recrystallize the precipitate with ethanol to give the compound 6-(2-hydroxy-ethylthio)-9-chloro-11H-indeno[1,2-c]quinolin-11-one; (5) dissolve 6,9-dichloro-11H-indeno[1,2-c]quinoline-11-one obtained from step 1 in dimethyl formamide and then add conc. hydrochloric acid at 150° C. for 24 hours. At the end of reaction, pour the mixture into ice water and incubate for 10 to 20 minutes. Finally, recrystallize the precipitate with ethanol to give the compound 6-hydroxy-9-chloro-11H-indeno[1,2-c]quinolin-11-one; (6) dissolve 6,9-dichloro-11H-indeno[1,2-c]quinoline-11-one obtained from step 1 in methanol then add sodium methoxide at 90° C. for 10 hours. At the end of reaction, cool the mixture and recrystallize the precipitate with ethanol to give the compound 6-methoxy-9-chloro-11H-indeno[1,2-c]quinolin-11-one.

These features and advantages of the present invention will be fully understood and appreciated from the following detailed description of the accompanying Drawings.

### BRIEF DESCRIPTION OF THE DRAWINGS

FIG. 1 shows the overview of the chemical reaction for synthesis of various compounds. Reactoin i is NaOAc at 200° C. for 2 hours, reaction ii is POCl3 at 150° C. for 48 hours, reaction iii: (a) SJ-1-3: the reactions of primary amines DMF and DIPEA in a mini-reactor at 150° C. for 2 hours; (b) SJ-4-24: reactions of secondary amines DMF and pyridine in a mini-reactor at 150° C. for 2 hours; (c) SJ-25: add 2-mercaptoethanol/K₂CO₃; (d) SJ-26: add conc. HCl, DMF, reflux 24 hours; (e) SJ-27: NaOMe, MeOH, reflux 8 hours.

FIG. 2 shows the screening results of 4 compounds selected from SRB assays for use as the Topoisomerase I drugs.

FIG. 3 shows the TC-XCl (NSC 773972) cytotoxicity in vitro against cancer cell lines National Cancer Institute (NCI)'s anticancer drug screen.

FIG. 4 shows the SJ-1 (NSC 772856) cytotoxicity in vitro against cancer cell lines National Cancer Institute (NCI)'s anticancer drug screen.

FIG. 5 shows the SJ-2 (NSC 771781) cytotoxicity in vitro against cancer cell lines National Cancer Institute (NCI)'s anticancer drug screen.

FIG. 6 shows the SJ-3 (NSC 772864) cytotoxicity in vitro against cancer cell lines National Cancer Institute (NCI)'s anticancer drug screen.

FIG. 7 shows the SJ-6 (NSC 772860) cytotoxicity in vitro against cancer cell lines National Cancer Institute (NCI)'s anticancer drug screen.

FIG. 8 shows the SJ-8 (NSC 771782)) cytotoxicity in vitro against cancer cell lines National Cancer Institute (NCI)'s anticancer drug screen.

FIG. 9 shows the SJ-9 (NSC 772857) cytotoxicity in vitro against cancer cell lines National Cancer Institute (NCI)'s anticancer drug screen.

FIG. 10 shows the SJ-10 (NSC 772862) cytotoxicity in vitro against cancer cell lines National Cancer Institute (NCI)'s anticancer drug screen.

FIG. 11 shows the SJ-12 (NSC 771783) cytotoxicity in vitro against cancer cell lines National Cancer Institute (NCI)'s anticancer drug screen.

FIG. 12 shows the SJ-14 (NSC 772859) cytotoxicity in vitro against cancer cell lines National Cancer Institute (NCI)'s anticancer drug screen.

FIG. 13 shows the SJ-16 (NSC 772861) cytotoxicity in vitro against cancer cell lines National Cancer Institute (NCI)'s anticancer drug screen.

FIG. 14 shows the SJ-20 (NSC 772863) cytotoxicity in vitro against cancer cell lines National Cancer Institute (NCI)'s anticancer drug screen.

FIG. 15 shows the SJ-23 (NSC 772858) cytotoxicity in vitro against cancer cell lines National Cancer Institute (NCI)'s anticancer drug screen.

FIG. 16 shows the SJ-25 (NSC 765596) cytotoxicity in vitro against cancer cell lines National Cancer Institute (NCI)'s anticancer drug screen.

### DETAILED DESCRIPTION OF THE PREFERRED EMBODIMENT

Unless defined otherwise, all technical and scientific terms used herein have the meaning commonly understood by a person skilled in the art to which this invention belongs. As used herein, the following terms have the meanings ascribed to them unless specified otherwise. The present invention will now be described more specifically with reference to the following embodiments, which are provided for the purpose of demonstration rather than limitation.

The term “treatment”, “under treatment” and similar terms refer to the methods which ameliorate, improve, reduce or reverse the patient's disease or any relevant symptoms caused by the disease, or methods which can prevent the onset of such diseases or any resulting symptoms.

The term ““pharmaceutically acceptable” is used to describe substances to be used in the composition which must be compatible with other ingredients in the formulation and be harmless to the subject.

The inventive composition can be prepared into a dosage form for suitable application of the inventive composition by using technology commonly understood by a person skilled in the art through formulating the abovementioned compound with a pharmaceutically acceptable vehicle, wherein the excipients include, but are not limited to, solution, emulsion, suspension, powder, tablet, pill, lozenge, troche, chewing gum, slurry, and other suitable forms.

The pharmaceutically acceptable vehicle may contain one or several reagents selected from the following list: solvents, emulsifiers, suspending agents, decomposers, binding agents, excipients, stabilizing agents, chelating agents, diluents, gelling agents, preservatives, lubricants, surfactants and other agents suitable for use in the invention.

In the abovementioned compositions, one or more dissolving aids, buffers, preservatives, colorants, fragrances, flavoring agents and the like, which are commonly used for formulation can be added as desired.

The term “pharmaceutically acceptable excipients”, include, but are not limited to, polymers, resins, plasticizers, fillers, lubricants, diluents, binders, disintegrants, solvents, co-solvents, surfactants, preservatives, sweetening agents, flavoring agents, pharmaceutical grade dyes or pigments, and viscosity agents.

The term “pharmaceutical composition” is used to describe solid or liquid compositions in a form, concentration and purity that is suitable for administration in patients (e.g. humans or animals) and can induce desired physiological changes following administration. Pharmaceutical compositions are typically sterile and non-pyrogenic.

The present invention will now be described more specifically with reference to the following embodiments, which are provided for the purpose of demonstration rather than limitation. The drugs as well as biomaterials used in the invention are all commercially available materials and the sources disclosed below are merely examples.

General Procedures for Chemical Synthesis

General procedure A: Synthesis of the compound TC-XCl-1. Mix isatin (0.44 g, 3 mmole), 2-(4-chlorophenyl)acetic acid (0.59 g, 3.47 mmole) and sodium acetate (0.05 g) in a miniclave (200° C.) for 3 hours. Allow cooling of the mixture before addition of acetic acid (10 mL) and collect precipitate by filtration and extraction. Wash the precipitate with acetic acid, water and n-hexane to obtain the intermediate product (TC-XCl).

Slowly add dried intermediate product (TC-XCl) (0.72 g, 2.4 mmole) to phosphoryl trichloride (20 mL) and allow to reflux at 150° C. for 48 hours. Upon completion of the reaction, allow cooling to room temperature followed by addition of 0° C. ice water. Collect precipitate by filtration and suction and add the collected precipitate to 10% sodium bicarbonate solution (300 mL) for 1 hr with vigorous shaking. Wash with water and recrystallize the crude product with dichloromethane to give the orange compound, TC-XCl-1.

General procedure B: Synthesis of the compounds SJ-1 and SJ-3. Dissolve 0.3 g TC-XCl-1 (1 mmole) in N,N-dimethylformamide (10 mL) and then add methylamine/N1,N1-diethylethane-1,2-diamine (10 mmole). Catalyze the reaction with 0.5 mL N,N-diisopropylethylamine (2.9 mmole) and place in a miniclave (150° C.) for 4 hours. At the end of reaction, pour the mixture into 100 mL ice water and incubate for 10 to 20 minutes to allow precipitation. Collect the precipitate by filtration and suction and recrystallize the precipitate with ethanol to obtain the compounds SJ-1 and SJ-3.

General procedure C: Synthesis of the compounds SJ-2, SJ4-24, 26 and 27. Dissolve 0.3 g of TC-XCl-1 (1 mmole) in 10 mL N,N-dimethylformamide and add secondary amine (10 mmole) and catalyze the reaction by adding 0.5 mL pyridine (6.21 mmole) and incubate in a miniclave (150° C.) for 4 hours. At the end of reaction, pour the mixture into 100 mL of ice water and incubate for 10 to 20 minutes to acquire precipitate. Vacuum extraction of the precipitate and recrystallize the precipitate with ethanol to obtain SJ-2 and SJ4-24.

General procedure D: Synthesis of the compound SJ-25. Dissolve 0.3 g of TC-XCl-1 (1 mmole) in 10 mL N,N-dimethylformamide (10 ml) and add 0.78 g of 2-mercaptoethanol (10 mmole) and catalyze the reaction by adding 0.27 g of potassium carbonate (2 mmole) and incubate in a miniclave (150° C.) for 4 hours. At the end of reaction, pour the mixture into 100 mL of ice water and incubate for 10 to 20 minutes to acquire precipitate. Vacuum extraction of the precipitate and recrystallize the precipitate with ethanol to obtain SJ-25.

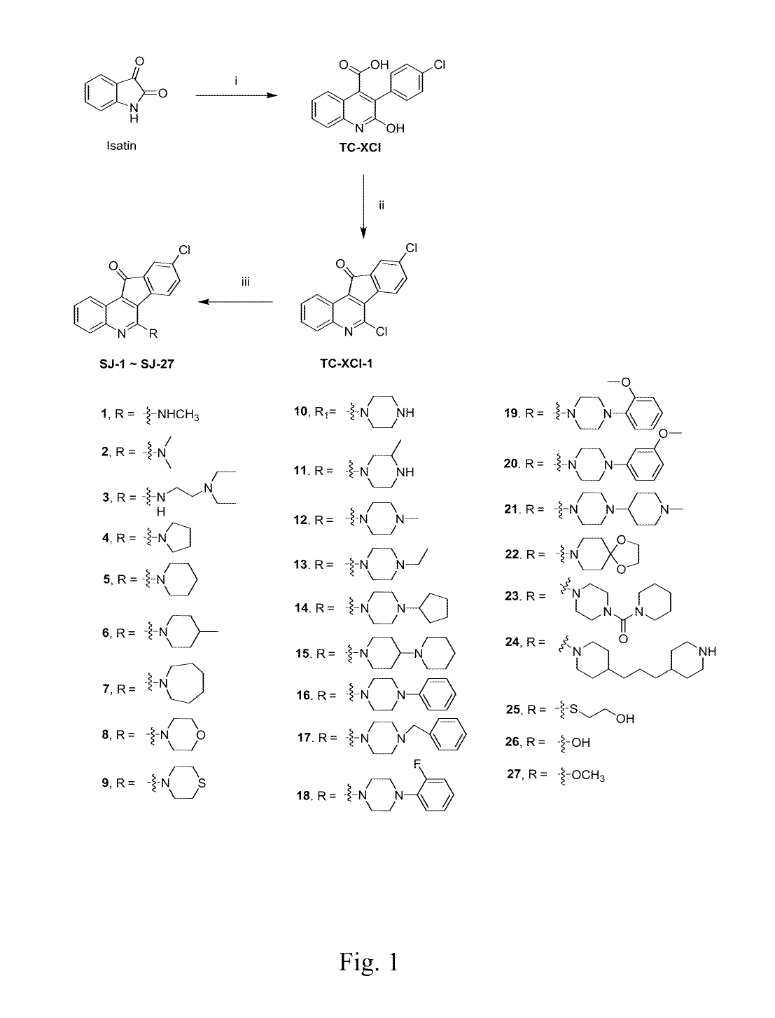

A total of 28 compounds were synthesized (FIG. 1) and are numbered as TC-XCl-1 and SJ-1 through SJ-27 including compound synthesis process, nomenclature, methods for synthesis, molecular formula, molecular weight, melting point, Rf, HRMS, IR, 1H-NMR, and 13C-NMR. The melting point was measured using a Biichi 545 melting point apparatus in our lab and the measurement results are accurate to the first decimal place. In order to ensure consistency, only the integer parts of the numbers were recorded. IR values are determined using Shimadzu FTIR-8300 infrared spectrometer which is located in Department of Pharmacy, National Defense Medical College on the ninth floor. 1H-NMR and 13C-NMR values are measure using the Varian GEMINI-300 (300 MHz) which is located in the basement of National Defense Medical Center. Determination of molecular weights was conducted at in the Department of Chemistry, National Tsing Hua University (Precious Instrument Center of NSC). MAT-95XL HRMS is measured using the high-resolution mass spectrometry (HRMS) (EI) Finnigan/Thermo Quest MAT HRMS in the Department of Chemistry, National Chung Hsing University, and a high-resolution electrospray ionization mass spectrometry HRMS (ESI) FINNIGAN MAT95S in the Department of Chemistry, National Taiwan University.

### Example 1

### 6,9-Dichloro-11H-indeno[1,2-c]quinolin-11-one (TC-XCl-1)

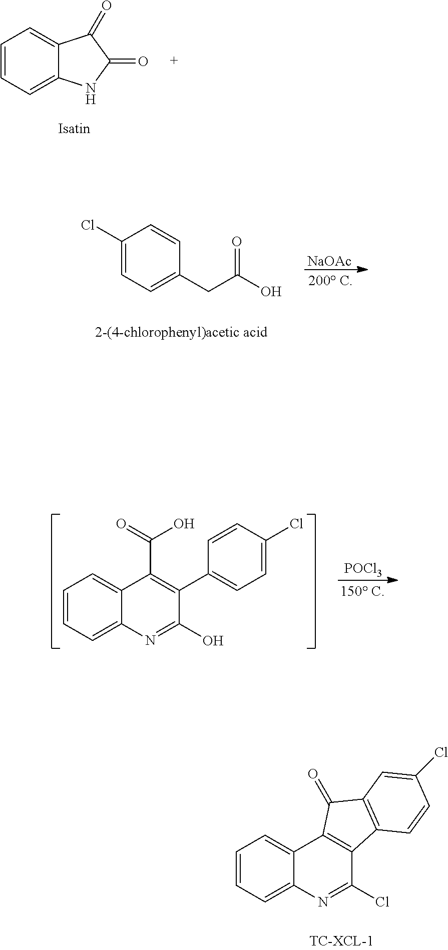

A mixture of isatin 0.44 g (3 mmol), 2-(4-chlorophenyl) acetic acid 0.59 g (3.47 mmol), and sodium acetate (0.05 g) was heated in miniclave at 200° C. for 3 hours (TLC monitoring). Add 10 mL of acetic acid to the mixture after cooling, and collect the precipitate was and wash with H₂O and n-hexane to get compound TC-XCl (0.72 g, 80%).

Add 3-(4-chlorophenyl)-2-hydroxyquinoline-4-carboxylic acid (0.72 g, 2.4 mmol) to 20 mL of phosphoryl trichloride and reflux the mixture at 150° C. for 48 hours. After cooling, pour the mixture into ice water (300 mL) at 0° C. Collect the resulting precipitate by filtration and mix with 10% NaHCO₃ solution (300 mL) with vigorous stirring for 1 hr. Wash the resulting precipitate with H₂O. The crude product was recrystallized from dichloromethane to get orange product TC-XCl-1 (0.22 g, 30.0%).

Mol. Wt.: 300.1389 (C₁₆H₇Cl₂NO); Yield: 30.0%; Mp.: 241-243° C. (EtOH); R_{f}: 0.48 (Dichloromethane:Hexane=2:1); IR (KBr) cm⁻¹: 1719 (C═O); HRMS (ESI) m/z calcd for C₁₆H₇Cl₂NO⁺[M]+: 298.9905. Found: [M+H]⁺=299.9965 (error<0.3%); ¹H-NMR (300 MHz, CDCl₃) δ (ppm): 7.52 (dd, J=8.25, 1.8 Hz, 1H, Ar—H₈), 7.62-7.68 (m, 2H, Ar—H_{2,10}), 7.70-7.76 (m, 1H, Ar—H₃), 7.97-8.01 (dt, J=7.5, 0.6 Hz, 1H, Ar—H₄), 8.10 (d, J=7.8 Hz, 1H, Ar—H₇), 8.77-8.80 (m, 1H, Ar—H₁); ¹³C-NMR (75 MHz, CDCl₃) δ (ppm): 122.97, 124.59, 125.30, 125.69, 129.13, 130.32, 131.70, 135.06, 135.14, 136.19, 136.74, 136.80, 140.15, 145.25, 150.48, 192.804 (CO).

### Example 2

### 9-Chloro-6-(methylamino)-11H-indeno[1,2-c]quinolin-11-one (SJ-1)

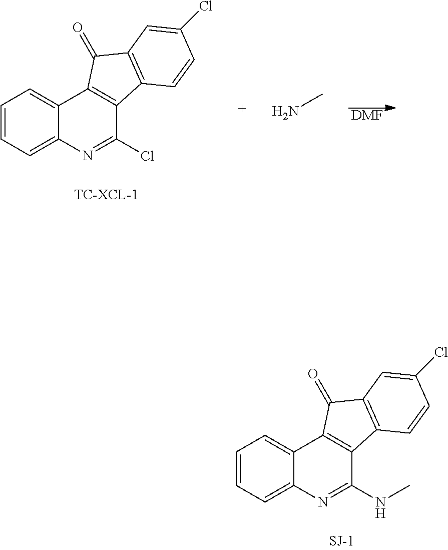

Add Methylamine (0.39 mL, 10 mmole) to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added 0.5 mL N,N-diisopropylethylamine (2.9 mmole) to catalyze the reaction. Pour the mixture into ice water after the reaction is completed and filter to get the crude product. Recrystallize the crude product from hot EtOH to get compound SJ-1 (0.22 g).

Mol. Wt.: 294.735 (C₁₇H₁₁ClN₂O); R_{f}: 0.51 (Dichloromethane:Hexane=2:1); Yield: 75.0%; Mp.: 189-191° C. (EtOH); IR (KBr) cm⁻¹: 1716 (C═O); HRMS (ESI) m/z calcd for C₁₇H₁₁ClN₂O⁺[M]⁺: 294.0560. Found: [M+H]⁺=295.0634 (error<0.3%); ¹H-NMR (300 MHz, CDCl₃) δ (ppm): 3.01 (s, 3H, N—CH₃), 7.41-7.47 (m, 2H, Ar—H_{8,10}), 7.57-7.62 (m, 3H, Ar—H_{2,3,7}), 7.84 (d, J=8.4 Hz, 1H, Ar—H₄), 8.68 (d, J=8.1 Hz, 1H, Ar—H₁); ¹³C-NMR (75 MHz, CDCl₃) δ (ppm): 42.22, 120.81, 124.29, 124.93, 124.99, 127.02, 128.13, 130.44, 131.60, 134.33, 135.16, 135.24, 136.59, 141.85, 149.69, 158.14, 194.48 (CO).

### Example 3

### 9-Chloro-6-(dimethylamino)-11H-indeno[1,2-c]quinolin-11-one (SJ-2)

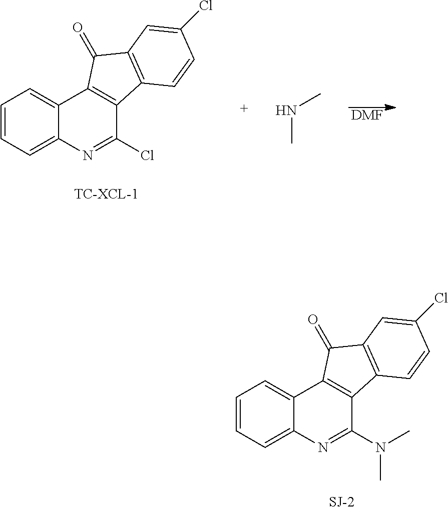

Dimethylamine (0.51 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours anded added pyridine (0.5 mL, 6.21 mmole) as the catalyst. Pour the mixture into ice water after the reaction is completed and filter to get the crude product. Recrystallized from hot EtOH to get compound SJ-2 (0.23 g).

Mol. Wt.: 308.7617 (C₁₈H₁₃ClN₂O); R_{f}: 0.51 (Dichloromethane:Hexane=2:1); Yield: 74.5%; Mp.: 193-195° C. (EtOH); IR (KBr) cm⁻¹: 3407 (N—H stretch), 1718 (C═O); HRMS (EI) m/z calcd for C₁₈H₁₃ClN₂O⁺[M]⁺: 308.0716. Found: 308.0708 (error<0.3%); ¹H-NMR (300 MHz, CDCl₃) δ (ppm): 3.04 (s, 6H, —N—(CH₃)₂), 7.41-7.48 (m, 2H, Ar—H_{8,10}), 7.57-7.63 (m, 3H, Ar—H_{2,3,7}), 7.86 (d, J=8.7 Hz, 1H, Ar—H₄), 8.68-8.71 (m, 1H, Ar—H₁); ¹³C-NMR (75 MHz, CDCl₃) δ (ppm): 42.19, 120.80, 124.27, 124.91, 124.96, 126.99, 128.13, 130.40, 131.57, 134.30, 135.14, 135.22, 136.54, 141.84, 149.70, 158.13, 194.45 (CO).

### Example 4

### 6-(2-(Diethylamino)ethylamino)-9-chloro-11H-indeno[1,2-c]quinolin-11-one (SJ-3)

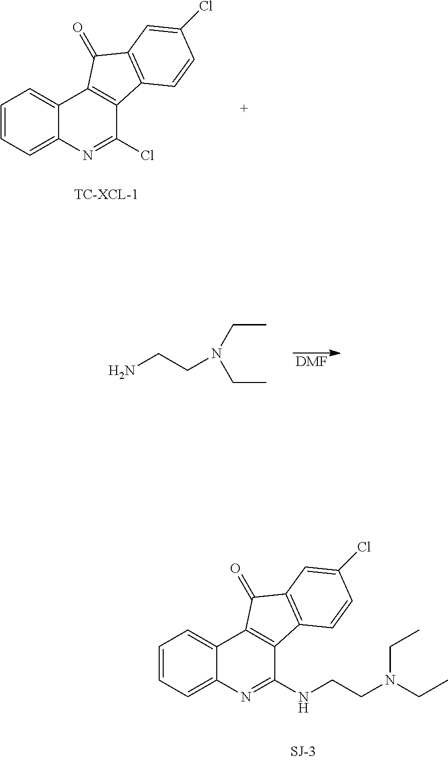

N¹,N¹-diethylethane-1,2-diamine (1.44 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 115° C. for 24 hours and added N,N-diisopropylethylamine (0.5 mL, 2.9 mmole) as catalyst. Pour the mixture into ice water after the reaction is completed and filter to get the crude product. Recrystallized from hot EtOH to get compound SJ-3 (0.07 g).

Mol. Wt.: 379.8826 (C₂₂H₂₂ClN₃O); R_{f}: 0.46 (Dichloromethane:Hexane=2:1); Yield: 17.5%; Mp.: 160-161° C. (EtOH); IR (KBr) cm⁻¹: 3371 (N—H stretch), 1712 (C═O); HRMS (ESI) m/z calcd for C₂₂H₂₂ClN₃O⁺[M]⁺: 379.1451. Found: [M+H]⁺=380.1510 (error<0.3%); ¹H-NMR (300 MHz, CDCl₃) δ (ppm): 1.11 (t, J=7.2 Hz, 6H, CH₃), 2.66 (q, J=7.1 Hz, 4H, —NCH₂—), 2.84 (t, J=5.7 Hz, 2H, —CH₂N—), 3.71-3.73 (m, 2H, NHCH₂—), 6.14 (br, 1H, NH), 7.28-7.33 (m, 1H, Ar—H₈), 7.41-7.55 (m, 3H, Ar—H_{2,3,10}), 7.60 (d, J=1.5 Hz, 1H, Ar—H₄), 7.70 (d, J=8.7 Hz, 1H, Ar—H₇), 8.60 (m, J=8.1 Hz, 1H, Ar—H₁); ¹³C-NMR (75 MHz, CDCl₃) δ (ppm): 12.23, 38.81, 46.92, 51.64, 119.00, 122.48, 124.42, 124.95, 125.40, 126.94, 127.86, 130.53, 134.07, 134.90, 135.08, 135.45, 141.24, 150.68, 152.96, 194.65 (CO).

### Example 5

### 9-Chloro-6-(pyrrolidin-1-yl)-11H-indeno[1,2-c]quinolin-11-one (SJ-4)

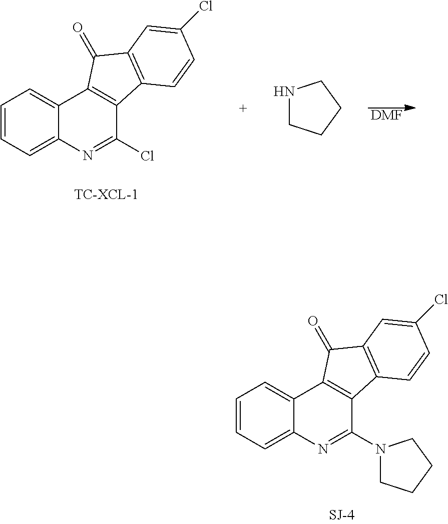

Add 0.83 mL of pyrrolidine (10 mmole) to DMF (10 mL) containing the compound 6,9-dichloro-11H-indeno[1,2-c]quinolin-11-one (TC-XCl-1) (0.3 g, 1 mmol). The mixture was heated while stirring at 150° C. for 4 hours and added 0.5 mL of pyridine (6.21 mmole) to catalyze the reaction. Pour the mixture into ice water after the reaction is completed and filter to get the crude product. Recrystallized from hot EtOH to get compound SJ-4 (0.15 g).

Mol. Wt.: 334.7989 (C₂₀H₁₅N₄ClN₂O); R_{f}: 0.51 (Dichloromethane:Hexane=2:1); Yield: 43.6%; Mp.: 149-150° C. (EtOH); IR (KBr) cm⁻¹: 1718 (C═O); HRMS (ESI) m/z calcd for C₂₀H₁₅N₄ClN₂O⁺[M]⁺: 334.0873. Found: [M+H]⁺=335.0952 (error<0.3%); ¹H-NMR (300 MHz, CDCl₃) δ (ppm): 1.98 (quin, J=3.6 Hz, 4H, H-3′,4′), 3.56 (t, J=6.6 Hz, 4H, H-2′,5′), 7.33-7.42 (m, 3H, Ar—H_{2,8,10}), 7.54 (td, J=7.5, 1.5 Hz, 2H, Ar—H_{3,4}), 7.76 (d, J=8.4 Hz, 1H, Ar—H₇), 8.63 (dd, J=8.4, 0.9 Hz, 1H, Ar—H₁); ¹³C-NMR (75 MHz, CDCl₃) δ (ppm): 25.07, 50.37, 120.39, 124.25, 124.80, 124.96, 126.29, 127.78, 130.33, 130.40, 134.10, 134.83, 135.27, 136.46, 142.50, 149.78, 155.73, 194.65 (CO).

### Example 6

### 9-Chloro-6-(piperidin-1-yl)-11H-indeno[1,2-c]quinolin-11-one (SJ-5)

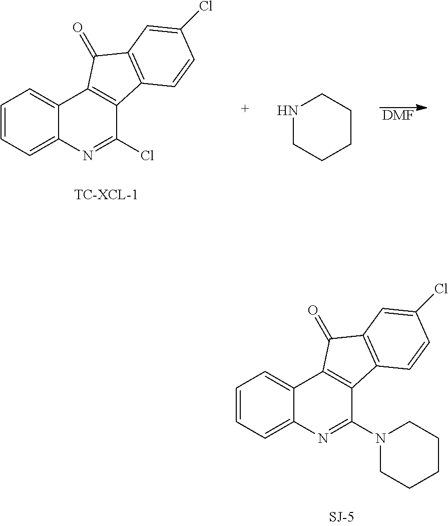

Add 0.99 mL of piperidine (10 mmole) to 10 mL of DMF containing 0.3 g of 6,9-dichloro-11H-indeno[1,2-c]quinolin-11-one (TC-XCl-1)(1 mmol). The mixture was heated while stirring at 150° C. for 4 hours followed by addition of 0.5 mL of pyridine (6.21 mmole) to catalyze the reaction. Pour the mixture into ice water after reaction and filter to get the crude product. Recrystallized from hot EtOH to get compound SJ-5 (0.15 g).

Mol. Wt.: 348.8255 (C₂₁H₁₇ClN₂O); R_{f}: 0.63 (Dichloromethane:Hexane=2:1); Yield: 43.0%; Mp.: 191-192° C. (EtOH); IR (KBr) cm⁻¹: 1717 (C═O); HRMS (ESI) m/z calcd for C₂₁H₁₇ClN₂O⁺[M]⁺: 348.1029. Found: [M+H]⁺=349.1106 (error<0.3%); ¹H-NMR (300 MHz, CDCl₃) δ (ppm): 1.71 (br, 2H, H-_{4′}), 1.83-1.86 (m, 4H, H-_{3′,5′}), 3.33 (br, 4H, H-_{2′,6′}), 7.43-7.48 (m, 2H, Ar—H_{8,10}), 7.58-7.66 (m, 3H, Ar—H_{2,3,4}), 7.90 (d, J=8.1 Hz, 1H, Ar—H₇), 8.70 (d, J=8.7 Hz, 1H, Ar—H₁); ¹³C-NMR (75 MHz, CDCl₃) δ (ppm): 24.37, 26.01, 51.30, 120.81, 124.22, 124.41, 124.83, 127.09, 128.29, 130.24, 131.88, 134.24, 135.01, 135.17, 136.35, 142.05, 149.79, 158.35, 194.38 (CO).

### Example 7

### 9-Chloro-6-(4-methylpiperidin-1-yl)-11H-indeno[1,2-c]quinolin-11-one (SJ-6)

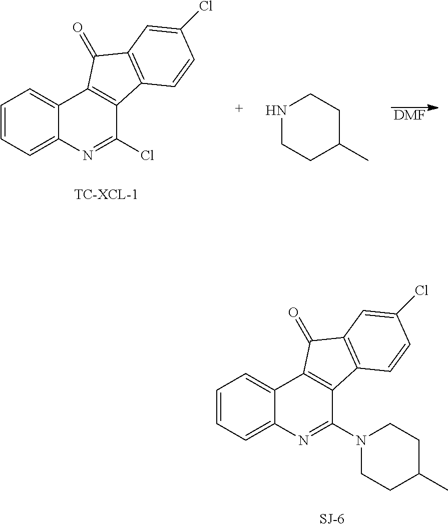

4-methylpiperidine (1.18 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-6 (0.09 g).

Mol. Wt.: 362.8521 (C₂₂H₁₉ClN₂O); R_{f}: 0.61 (Dichloromethane:Hexane=2:1); Yield: 25.4%; Mp.: 190-192° C. (EtOH); IR (KBr) cm⁻¹: 1718 (C═O); HRMS (ESI) m/z calcd for C₂₂H₁₉ClN₂O⁺[M]⁺: 362.1186. Found: [M+H]⁺=363.1260 (error<0.3%); ¹H-NMR (300 MHz, CDCl₃) δ (ppm): 1.07 (d, J=6 Hz, 3H, —CH₃), 1.46-1.60 (m, 3H, —CH₂—, —CH—), 1.84-1.87 (m, 2H, —CH₂—), 2.96 (t, J=11.6 Hz, 2H, N—CH₂—), 3.67-3.71 (m, 2H, N—CH₂—), 7.44-7.46 (m, 2H, Ar—H_{8,10}), 7.58 (m, 3H, Ar—H_{2,3,4}), 7.82-7.85 (m, 1H, Ar—H₇), 8.66-8.69 (m, 1H, Ar—H₁); ¹³C-NMR (75 MHz, CDCl₃) δ (ppm): 22.07, 30.87, 34.43, 50.69, 120.88, 124.27, 124.46, 124.91, 127.15, 128.31, 130.32, 131.98, 134.33, 135.07, 135.23, 136.44, 142.13, 149.83, 158.25, 194.52 (CO).

### Example 8

### 6-(Azepan-1-yl)-9-chloro-11H-indeno[1,2-c]quinolin-11-one (SJ-7)

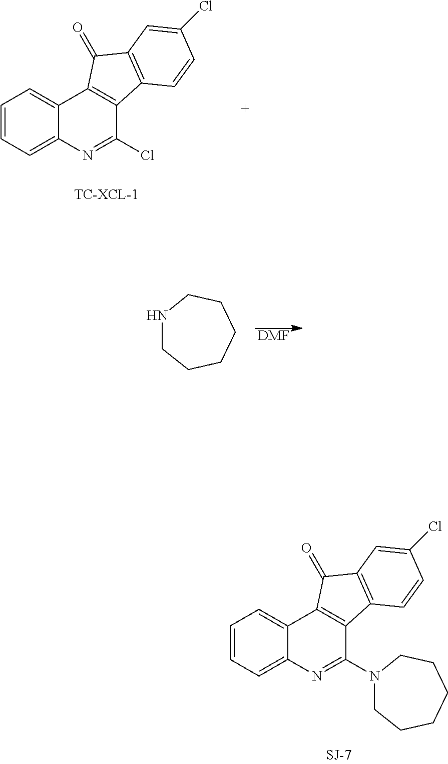

Azepane (1.13 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-7 (0.13 g).

Mol. Wt.: 362.8521 (C₂₂H₁₉ClN₂O); R_{f}: 0.69 (Dichloromethane:Hexane=2:1); Yield: 35.8%; Mp.: 146-147° C. (EtOH); IR (KBr) cm⁻¹: 1712 (C═O); MS (ESI) m/z calcd for C₂₂H₁₉ClN₂O⁺[M]⁺: 362.1186. Found: [M+H]⁺=363.2000 (error<0.3%); ¹H-NMR (300 MHz, CDCl₃) δ (ppm): 1.72-1.75 (m, 4H, —CH₂—), 1.86 (br, 4H, —CH₂—), 3.64 (t, J=5.6 Hz, 4H, —N—CH₂—), 7.40-7.46 (m, 2H, Ar—H_{8,10}), 7.54-7.60 (m, 3H, Ar—H_{2,3,4}), 7.78-7.80 (m, 1H, Ar—H₇), 8.68 (dd, J=8.4, 0.6 Hz, 1H, Ar—H₁); ¹³C-NMR (75 MHz, CDCl₃) δ (ppm): 27.96, 28.40, 52.91, 120.48, 124.24, 124.76, 124.89, 126.60, 127.98, 130.30, 131.09, 134.13, 134.97, 135.15, 136.84, 142.54, 149.74, 157.83, 194.62 (CO).

### Example 9

### 9-Chloro-6-morpholino-11H-indeno[1,2-c]quinolin-11-one (SJ-8)

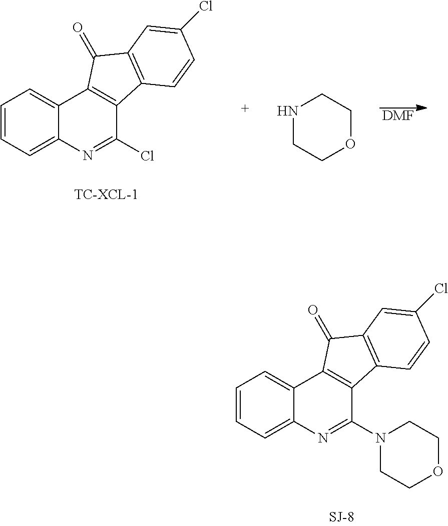

Morpholine (0.86 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-8 (0.16 g).

Mol. Wt.: 350.7983 (C₂₀H₁₅aN₂O₂); R_{f}: 0.54 (Dichloromethane:Hexane=2:1); Yield: 47.0%; Mp.: 207-208° C. (EtOH); IR (KBr) cm⁻¹: 1712 (C═O); HRMS (ESI) m/z calcd for C₂₀H₁₅ClN₂O₂⁺[M]⁺: 350.0822. Found: [M+H]⁺=351.0898 (error<0.3%); ¹H-NMR (300 MHz, CDCl₃) δ (ppm): 3.41 (t, J=4.5 Hz, 4H, —CĤ₂—), 3.98 (t, J=4.5 Hz, 4H, —CĤ₂—), 7.48 (td, J=8.1, 2.1 Hz, 2H, Ar—H_{8,10}), 7.59-7.65 (m, 3H, Ar—H_{2,3,4}), 7.87 (d, J=8.7 Hz, 1H, Ar—H₇), 8.69-8.72 (dt, J=8.1, 0.9 Hz, 1H, Ar—H₁); ¹³C-NMR (75 MHz, CDCl₃) δ (ppm): 50.56, 66.98, 121.18, 124.35, 124.43, 125.23, 127.65, 128.51, 130.59, 131.46, 134.42, 135.21, 135.55, 136.80, 141.66, 149.81, 157.31, 194.14 (CO).

### Example 10

### 9-Chloro-6-thiomorpholino-11H-indeno[1,2-c]quinolin-11-one (SJ-9)

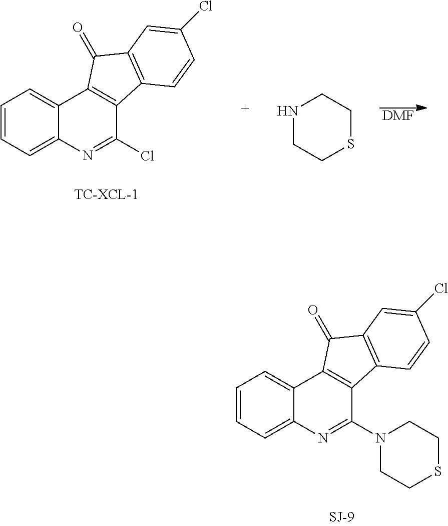

Thiomorpholine (1 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-9 (0.27 g).

Mol. Wt.: 366.8639 (C₂₀H₁₅ClN₂O₂); R_{f}: 0.33 (Dichloromethane:Hexane=2:1); Yield: 74.3%; Mp.: 228-230° C. (EtOH); IR (KBr) cm⁻¹: 1711 (C═O); HRMS (ESI) m/z calcd for C₂₀H₁₅ClN₂O₂⁺[M]⁺: 366.0594. Found: [M+H]⁺=367.0664 (error<0.3%); ¹H-NMR (300 MHz, CDCl₃) δ (ppm): 2.91 (t, J=5.1 Hz, 4H, —CH₂—), 2.69-2.73 (br, 4H, —CH₂—), 7.45-7.50 (td, J=7.8, 1.8 Hz, 2H, Ar—H_{8,10}), 7.57-7.64 (m, 3H, Ar—H_{2,3,4}), 7.85 (d, J=9.0 Hz, 1H, Ar—H₇), 8.68-8.71 (dd, J=8.25, 1.2 Hz, 1H, Ar—H₁); ¹³C-NMR (75 MHz, CDCl₃) δ (ppm): 27.38, 52.36, 121.07, 124.29, 125.19, 127.66, 128.48, 129.44, 130.57, 131.59, 134.40, 135.10, 135.54, 136.87, 141.70, 149.70, 157.66, 194.17 (CO).

### Example 11

### 9-Chloro-6-(piperazin-1-yl)-11H-indeno[1,2-c]quinolin-11-one (SJ-10)

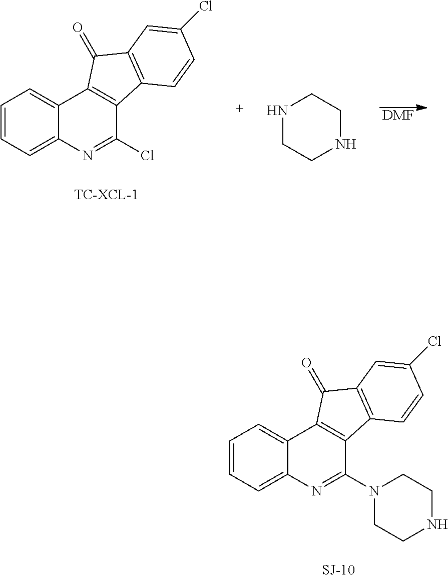

piperazine (0.86 g, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-10 (0.17 g).

Mol. Wt.: 349.8135 (C₂₀H₁₆ClN₃O); R_{f}: 0.43 (Dichloromethane:Hexane=2:1); Yield: 47.5%; Mp.: 180-181° C. (EtOH); R (KBr) cm⁻¹: 3341 (N—H stretch), 1718 (C═O); FIRMS (ESI) m/z calcd for C₂₀H₁₆ClN₃O⁺[M]⁺: 349.0982. Found: [M+H]⁺=350.1063 (error<0.3%); ¹H-NMR (300 MHz, CDCl₃) δ (ppm): 3.16 (t, J=4.8 Hz, 4H, —CH₂—), 3.36 (br, 4H, —CH₂—), 7.46-7.49 (m, 2H, Ar—H_{8,10}), 7.62-7.66 (m, 3H, Ar—H_{2,3,4}), 7.87 (d, J=8.7 Hz, 1H, Ar—H₇), 8.71 (d, J=8.7 Hz, 1H, Ar—H₁); ¹³C-NMR (75 MHz, CDCl₃) δ (ppm): 46.17, 51.51, 121.05, 124.31, 124.54, 125.11, 127.45, 128.46, 130.48, 131.69, 134.39, 135.16, 135.40, 136.68, 141.88, 149.84, 157.83, 194.37 (CO).

### Example 12

### 9-Chloro-6-(2-methylpiperazin-1-yl)-11H-indeno[1,2-c]quinolin-11-one (SJ-11)

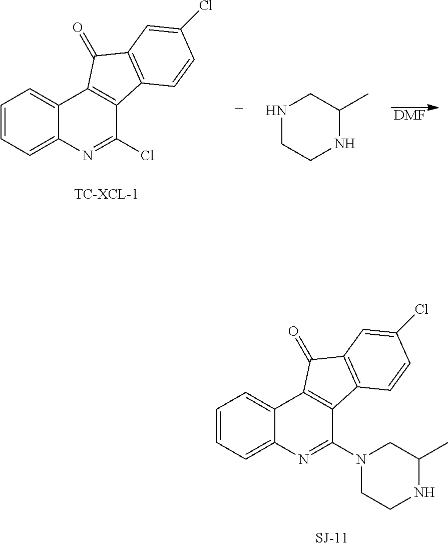

3-methylpiperazine (1.0 g, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-11 (0.06 g).

Mol. Wt.: 363.8401 (C₂₁H₁₈ClN₃O); R_{f}: 0.49 (Dichloromethane:Methanol=2:0.5); Yield: 17.5%; Mp.: 199-200° C. (EtOH); IR (KBr) cm⁻¹: 3222 (N—H stretch), 1719 (C═O); HRMS (ESI) m/z calcd for C₂₁H₁₈ClN₃O⁺[M]⁺: 363.1138. Found: [M+H]⁺=364.1201 (error<0.3%); ¹H-NMR (300 MHz, CDCl₃) δ (ppm): 1.17 (d, J=6.3 Hz, 3H, —CH₃), 2.70 (t, 1H, —CH₂—), 3.03-3.07 (m, 1H, N—CH—), 3.15-3.19 (m, 3H, —CH₂—, —CH₂—NH), 3.60-3.65 (d, J=12.6 Hz, 2H, NH—CH₂—), 7.44-7.48 (m, 2H, Ar—H_{8,10}), 7.58-7.62 (m, 3H, ArH_{2,3,4}), 7.86 (d, J=8.4 Hz, 1H, Ar—H₇), 8.69 (d, J=7.8 Hz, 1H, Ar—H₁); ¹³C-NMR (75 MHz, CDCl₃) δ (ppm): 19.96, 45.98, 50.63, 50.75, 57.67, 120.98, 124.29, 124.47, 125.09, 127.39, 128.41, 130.47, 131.63, 134.37, 135.15, 135.36, 136.65, 141.89, 149.82, 157.56, 194.36 (CO).

### Example 13

### 9-Chloro-6-(4-methylpiperazin-1-yl)-11H-indeno[1,2-c]quinolin-11-one (SJ-12)

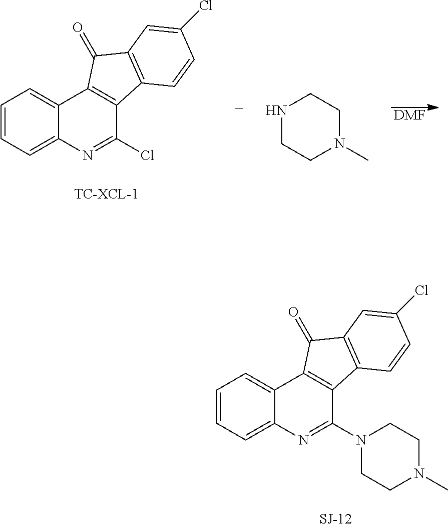

1-methylpiperazine (1.11 mL, 10 mmole) was added to 10 mL DMF containing 0.3 g of 6,9-dichloro-11H-indeno[ 1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-12 (0.19 g).

Mol. Wt.: 363.8401 (C₂₁H₁₈ClN₃O); R_{f}: 0.4 (Dichloromethane:Hexane=2:1); Yield: 51.4%; Mp.: 205-207° C. (EtOH); IR (KBr) cm⁻¹: 3426 (N—H stretch), 1720 (C═O); HRMS (ESI) m/z calcd for C₂₁H₁₈ClN₃O⁺[M]⁺: 363.1138. Found: [M+H]⁺=364.1222 (error<0.3%); ¹H-NMR (300 MHz, CDCl₃) δ (ppm): 2.42 (s, 3H, N—CH₃), 2.70 (br, 4H, —CH₂—), 3.43 (br, 4H, N—CH₂—), 7.44-7.47 (m, 2H, Ar—H_{8,10}), 7.58-7.61 (m, 3H, Ar—H_{2,3,4}), 7.84 (d, J=8.4 Hz, 1H, Ar—H₇), 8.67 (d, J=8.1 Hz, 1H, Ar—H₁); ¹³C-NMR (75 MHz, CDCl₃) δ (ppm): 46.38, 49.97, 55.17, 121.00, 124.28, 124.54, 125.07, 127.38, 128.46, 130.45, 131.51, 134.35, 135.13, 135.38, 136.63, 141.84, 149.80, 157.37, 194.29 (CO).

### Example 14

### 9-Chloro-6-(4-ethylpiperazin-1-yl)-11H-indeno[1,2-c]quinolin-11-one (SJ-13)

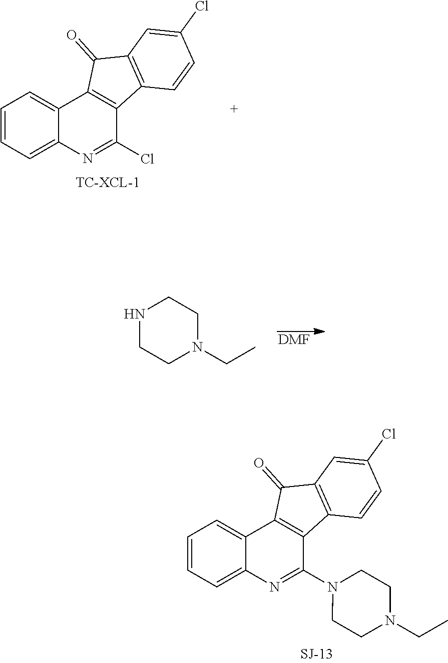

1-ethylpiperazine (1.27 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-13 (0.07 g).

Mol. Wt.: 377.8667 (C₂₂H₂₀ClN₃O); R_{f}: 0.43 (Dichloromethane:Hexane:Methanol=2:1:0.5); Yield: 19.8%; Mp.: 182-184° C. (EtOH); IR (KBr) cm⁻¹: 1710 (C═O); HRMS (ESI) m/z calcd for C₂₂H₂₀ClN₃O⁺[M]⁺: 377.1295. Found: [M+H]⁺=378.1380 (error<0.3%); ¹H-NMR (300 MHz, CDCl₃) δ (ppm): 1.19 (t, 3H, J=7.2 Hz, —CH₃), 2.57 (q, 2H, J=7.4 Hz, —N—CH₂—), 3.03 (br, 4H, —CH₂—), 3.46 (br, 4H, —CH₂—), 7.43-7.48 (m, 2H, Ar—H_{8,10}), 7.57-7.60 (m, 3H, Ar—H_{2,3,4}), 7.85 (d, J=8.4 Hz, 1H, Ar—H₇), 8.69 (dd, J=8.25, 0.9 Hz, 1H, Ar—H₁); ¹³C-NMR (75 MHz, CDCl₃) δ (ppm): 12.06, 49.99, 52.69, 52.85, 121.02, 124.30, 124.59, 125.07, 127.36, 128.49, 130.43, 131.53, 134.36, 135.19, 135.38, 136.64, 141.89, 149.84, 15740, 194.32 (CO).

### Example 15

### 9-Chloro-6-(4-cyclopentylpiperazin-1-yl)-11H-indeno[1,2-c]quinolin-11-one (SJ-14)

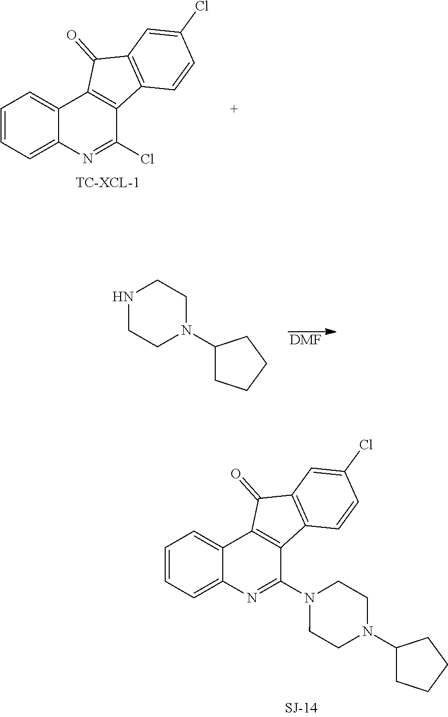

1-cyclopentylpiperazine (1.27 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-14 (0.16 g).

Mol. Wt.: 417.9306 (C₂₅H₂₄ClN₃O); R_{f}: 0.46 (Dichloromethane:Hexane:Methanol=2:1:0.5); Yield: 37.3%; Mp.: 183-184° C. (EtOH); IR (KBr) cm⁻¹: 1716 (C═O); HRMS (ESI) m/z calcd for C₂₅H₂₄ClN₃O⁺[M]⁺: 417.1608. Found: [M+H]⁺=418.1689 (error<0.3%); ¹H-NMR (300 MHz, CDCl₃) δ (ppm): 1.19 (t, 3H, J=7.2 Hz, —CH₃), 2.57 (q, 2H, J=7.4 Hz, N—CH₂—), 3.03 (br, 4H, —CH₂—), 3.46 (br, 4H, —CH₂—), 7.43-7.48 (m, 2H, Ar—H_{8,10}), 7.57-7.63 (m, 3H, Ar—H_{2,3,4}), 7.85 (d, J=8.7 Hz, 1H, Ar—H₇), 8.69 (d, J=8.1 Hz, 1H, Ar—H₁); ¹³C-NMR (300 MHz, CDCl₃) δ (ppm): 24.32, 30.67, 50.12, 52.36, 67.87, 120.99, 124.28, 124.61, 125.05, 127.31, 128.50, 130.40, 131.56, 134.36, 135.19, 135.32, 136.58, 141.94, 149.85, 157.48, 194.40 (CO).

### Example 16

### 9-Chloro-6-(4-(piperidin-1-yl)piperidin-1-yl)-11H-indeno[1,2-c]quinolin-11-one (SJ-15)

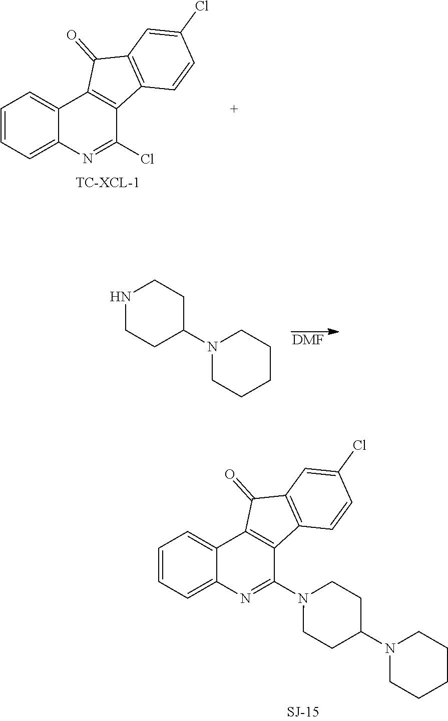

1-(piperidin-4-yl)piperidine (1.68 g, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-15 (0.25 g).

Mol. Wt.: 431.9571 (C₂₆H₂₆ClN₃O); R_{f}: 0.51 (Dichloromethane:Hexane:Methanol=2:1:0.5); Yield: 57.3%; Mp.: 174-175° C. (EtOH); IR (KBr) cm⁻¹: 1718 (C═O); HRMS (ESI) m/z calcd for C₂₆H₂₆ClN₃O⁺[M]⁺: 431.1764. Found: [M+H]⁺=432.1822 (error<0.3%); ¹H-NMR (300 MHz, CDCl₃) δ (ppm): 1.48-1.50 (m, 2H, —CH₂—), 1.63-1.65 (m, 2H, —CH₂—), 1.72-1.85 (m, 4H, —CH₂—), 2.08 (d, J=11.4 Hz, 2H, —CH₂—), 2.38-2.46 (m, 1H, —CH₂—), 2.60 (s, 4H, —CH—), 2.91-3.02 (m, 2H, —CH₂—), 3.76 (d, J=12.3 Hz, 2H, —CH₂—), 7.41-7.45 (m, 2H, Ar—H_{8,10}), 7.55-7.61 (m, 3H, Ar—H_{2,3,4}), 7.82 (d, J=8.4 Hz, 1H, Ar—H₇), 8.66 (d, J=7.8 Hz, 1H, Ar—H₁); ¹³C-NMR (300 MHz, CDCl₃) δ (ppm): 24.99, 26.61, 28.77, 50.17, 50.85, 62.51, 120.98, 124.27, 124.42, 124.96, 127.26, 128.36, 130.34, 131.86, 134.37, 135.08, 135.32, 136.45, 141.99, 149.80, 157.82, 194.39 (CO).

### Example 17

### 9-Chloro-6-(4-phenylpiperazin-1-yl)-11H-indeno[1,2-c]quinolin-11-one (SJ-16)

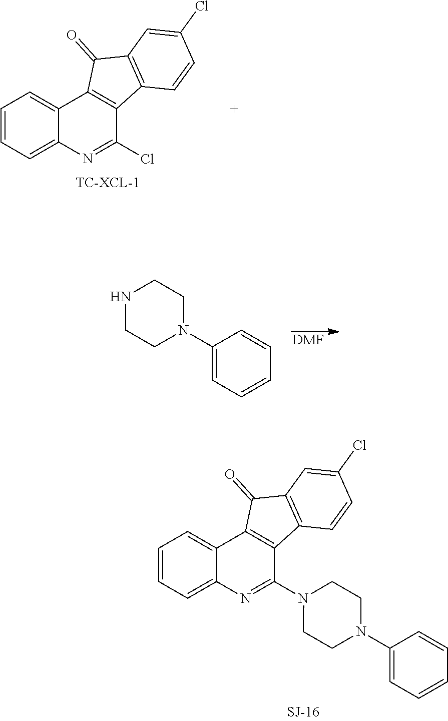

1-phenylpiperazine (1.53 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-16 (0.22 g).

Mol. Wt.: 425.9095 (C₂₆H₂₀ClN₃O); R_{f}: 0.91 (Dichloromethane:Hexane:Methanol=3:1:0.5); Yield: 51.7%; Mp.: 193-194° C. (EtOH); IR (KBr) cm⁻¹: 1714 (C═O); HRMS (ESI) m/z calcd for C₂₆H₂₀ClN₃O⁺[M]⁺: 425.1295. Found: [M+H]⁺=426.1370 (error<0.3%); ¹H-NMR (300 MHz, CDCl₃) δ (ppm): 3.47 (br, 4H, —CH₂—), 3.57 (br, 4H, —CH₂—), 6.93 (t, J=7.2 Hz, 1H, Ar—H), 7.04 (d, J=7.8 Hz, 2H, Ar—H), 7.30-7.36 (m, 2H, Ar—H), 7.45-7.51 (m, 2H, Ar—H_{8,10}), 7.58-7.70 (m, 3H, Ar—H_{2,3,4}), 7.88 (d, J=7.8 Hz, 1H, Ar—H₇), 8.72 (d, J=8.1 Hz, 1H, Ar—H₁); ¹³C-NMR (300 MHz, CDCl₃) δ (ppm): 49.44, 50.16, 116.66, 120.53, 121.14, 124.32, 124.52, 125.21, 127.60, 128.49, 129.57, 130.56, 131.60, 134.43, 135.16, 135.49, 136.68, 141.74, 149.78, 151.65, 157.41, 194.30 (CO).

### Example 18

### 6-(4-Benzylpiperazin-1-yl)-9-chloro-11H-indeno[1,2-c]quinolin-11-one (SJ-17)

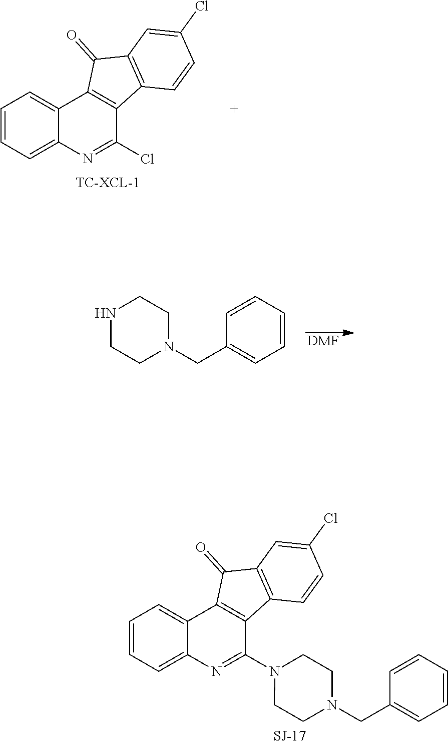

1-benzylpiperazine (1.74 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-17 (0.18 g).

Mol. Wt.: 439.9361 (C₂₇H₂₂ClN₃O); R_{f}: 0.37 (Dichloromethane:Hexane=2:1); Yield: 40.9%; Mp.: 178-180° C. (EtOH); IR (KBr) cm⁻¹: 1718 (C═O); HRMS (ESI) m/z calcd for C₂₇H₂₂ClN₃O⁺[M]⁺: 439.1451. Found: [M+H]⁺=440.1503 (error<0.3%); ¹H-NMR (300 MHz, CDCl₃) δ (ppm): 2.73 (br, 4H, —CH₂—), 3.41 (br, 4H, —CH₂—), 3.65 (s, 2H, —CH₂—) 7.28-7.47 (m, 7H, Ar—H_{8,10}, Ar—H), 7.57-7.63 (m, 3H, Ar—H_{2,3,4}), 7.84 (d, J=8.7 Hz, 1H, Ar—H₇), 8.68 (d, J=7.05 Hz, 1H, Ar—H₁); ¹³C-NMR (300 MHz, CDCl₃) δ (ppm): 50.11, 53.21, 63.34, 120.97, 124.26, 124.60, 125.04, 127.34, 127.50, 128.42, 128.64, 129.40, 130.42, 131.61, 134.35, 135.11, 135.30, 136.53, 138.46, 141.83, 149.78, 157.55, 194.38 (CO).

### Example 19

### 9-Chloro-6-(4-(2-fluorophenyl)piperazin-1-yl)-11H-indeno[1,2-c]quinolin-11-one (SJ-18)

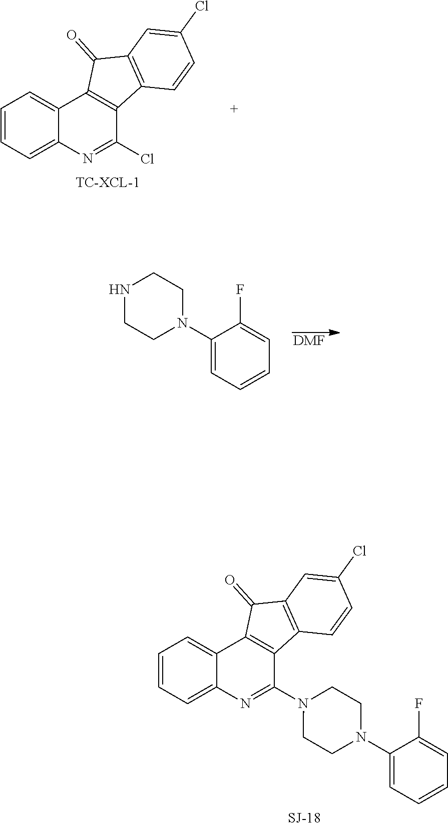

1-(2-fluorophenyl)piperazine (1.58 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as catalyst. Pour the mixture into ice water after the reaction is completed and filter to get the crude product. Recrystallized from hot EtOH to get compound SJ-18 (0.18 g).

Mol. Wt.: 443.9 (C₂₆H₁₉ClN₃O); R_{f}: 0.46 (Dichloromethane:Hexane=2:1); Yield: 40.6%; Mp.: 182-183° C. (EtOH); IR (KBr) cm⁻¹: 1715 (C═O); HRMS (ESI) m/z calcd for C₂₆H₁₉ClN₃O⁺[M]⁺: 443.1201. Found: [M+H]⁺=444.1269 (error<0.3%); ¹H-NMR (300 MHz, CDCl₃) δ (ppm): 3.37 (br, 4H, —CH₂—), 3.59 (br, 4H, —CH₂—), 7.00-7.12 (m, 4H, Ar—H), 7.44-7.50 (m, 2H, Ar—H_{8,10}), 7.59-7.68 (m, 3H, Ar—H_{2,3,4}), 7.88 (d, J=8.1 Hz, 1H, Ar—H₇), 8.71 (d, J=8.1 Hz, 1H, Ar—H₁); ¹³C-NMR (300 MHz, CDCl₃) δ (ppm): 50.27, 50.63, 116.47, 116.75, 119.48, 121.09, 123.06, 123.17, 124.31, 124.53, 124.80, 124.86, 125.17, 127.54, 128.49, 130.54, 131.55, 134.40, 135.16, 135.45, 141.76, 149.78, 157.37, 194.29 (CO).

### Example 20

### 9-Chloro-6-(4-(2-methoxyphenyl)piperazin-1-yl)-11H-indeno[1,2-c]quinolin-11-one (SJ-19)

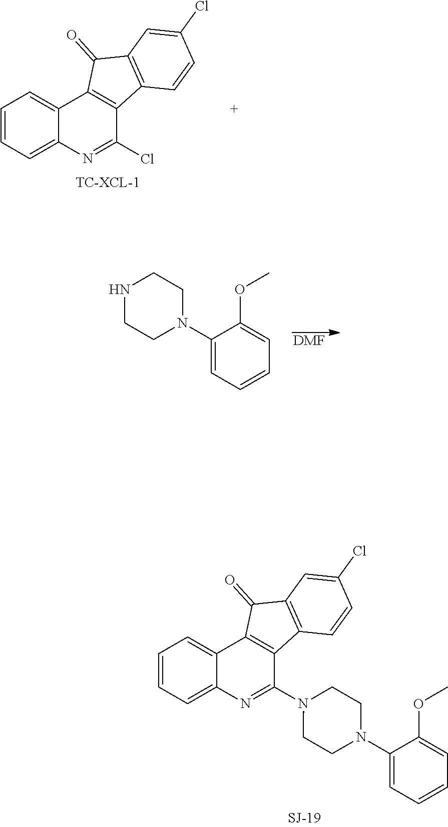

1-(2-methoxyphenyl)piperazine (1.38 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-19 (0.17 g).

Mol. Wt.: 455.9355 (C₂₇H₂₂ClN₃O₂); R_{f}: 0.38 (Dichloromethane:Hexane=2:1); Yield: 37.3%; Mp.: 129-131° C. (EtOH); IR (KBr) cm⁻¹: 1714 (C═O); HRMS (ESI) m/z calcd for C₂₇H₂₂ClN₃O₂⁺[M]⁺: 455.1401. Found: [M+H]⁺=456.1473 (error<0.3%); ¹H-NMR (300 MHz, CDCl₃) δ (ppm): 3.36 (br, 4H, —CH₂—), 3.60 (br, 4H, —CH₂—), 3.90 (s, 3H, —O—CH₃), 6.91-7.07 (m, 4H, Ar—H), 7.47 (t, J=7.5 Hz, 2H, Ar—H_{8,10}), 7.59-7.63 (m, 2H, Ar—H_{2,3}), 7.68 (d, J=7.8 Hz, 1H, Ar—H₄), 7.87 (d, J=8.1 Hz, 1H, Ar—H₇), 8.70 (d, J=8.4 Hz, 1H, Ar—H₁); ¹³C-NMR (300 MHz, CDCl₃) δ (ppm): 50.39, 50.75, 55.75, 112.15, 118.68, 121.03, 121.49, 123.55, 124.28, 124.61, 125.09, 127.40, 128.47, 130.46, 131.58, 134.36, 135.15, 135.36, 136.61, 141.66, 141.85, 149.81, 152.86, 157.53, 194.38 (CO).

### Example 21

### 9-Chloro-6-(4-(3-methoxyphenyl)piperazin-1-yl)-11H-indeno[1,2-c]quinolin-11-one (SJ-20)

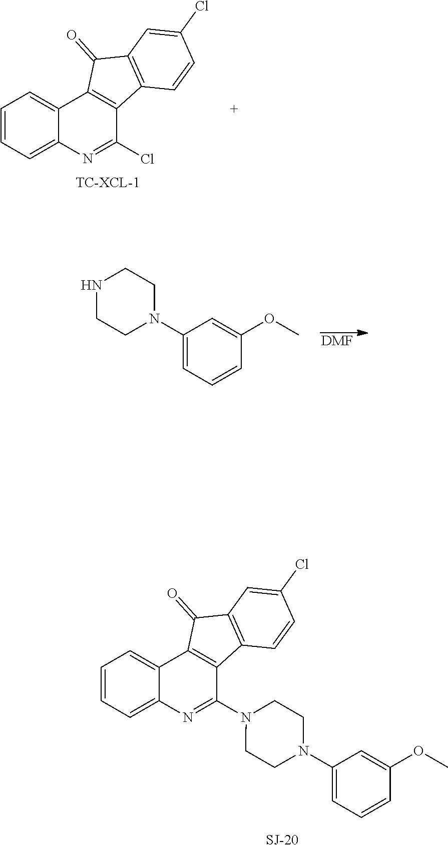

1-(3-methoxyphenyl)piperazine (1.73 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as catalyst. After reaction, poured it into ice water and filtered to gain crude. Recrystallized from hot EtOH to gain compound SJ-20 (0.39 g).

Mol. Wt.: 455.9355 (C₂₇H₂₂ClN₃O₂); R_{f}: 0.43 (Dichloromethane:Hexane=2:1); Yield: 85.9%; Mp.: 189-191° C. (EtOH); IR (KBr) cm⁻¹: 1723 (C═O); HRMS (ESI) m/z calcd for C₂₇H₂₂ClN₃O₂⁺[M]⁺: 455.1401. Found: [M+H]⁺=456.1464 (error<0.3%); ¹H-NMR (300 MHz, CDCl₃) δ (ppm): 3.46 (br, 4H, —CH₂—), 3.55 (br, 4H, —CH₂—), 3.83 (s, 3H, —O—CH₃), 6.50 (m, J=8.1 Hz, 1H, Ar—H), 6.57 (s, 1H, Ar—H), 6.65 (d, J=8.4 Hz, 1H, Ar—H), 7.22 (d, J=8.1 Hz, 1H, Ar—H), 7.44-7.51 (m, 2H, Ar—H_{8,10}), 7.60-7.68 (m, 3H, Ar—H_{2,3,4}), 7.88 (d, J=8.1 Hz, 1H, Ar—H₇), 8.71 (d, J=7.2 Hz, 1H, Ar—H₁); ¹³C-NMR (300 MHz, CDCl₃) δ (ppm): 49.36, 50.08, 55.46, 103.25, 105.29, 109.41, 121.14, 124.31, 124.50, 125.18, 127.60, 128.49, 130.25, 130.56, 131.56, 134.42, 135.15, 135.49, 136.68, 141.70, 149.77, 153.00, 157.35, 161.17, 194.23 (CO).

### Example 22

### 9-Chloro-6-(4-(1-methylpiperidin-4-yl)piperazin-1-yl)-11H-inden o[1,2-c]quinolin-11-one (SJ-21)

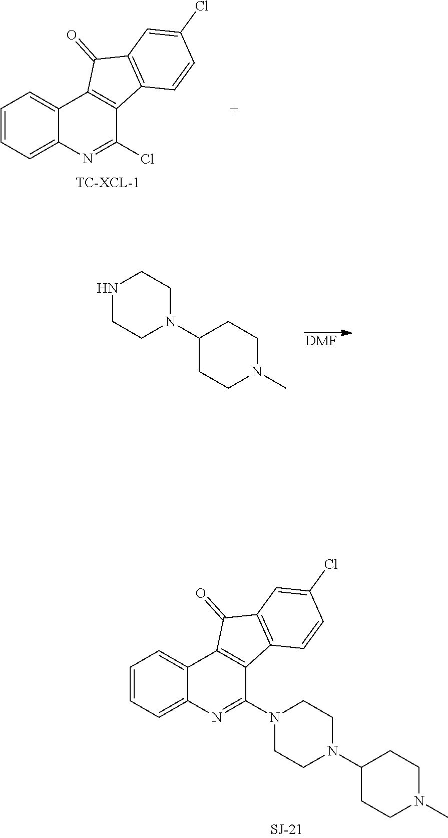

1-(1-methylpiperidin-4-yl)piperazine (1.83 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-21 (0.14 g).

Mol. Wt.: 446.9718 (C₂₆H₂₇ClN₄O); R_{f}: 0.90 (Dichloromethane:Hexane:Methanol=2:1:0.5); Yield: 30.4%; Mp.: 208-209° C. (EtOH); IR (KBr) cm⁻¹: 1710 (C═O); HRMS (ESI) m/z calcd for C₂₆H₂₇ClN₄O⁺[M]⁺: 446.1873. Found: [M+H]⁺=447.1944 (error<0.3%); ¹H-NMR (300 MHz, CDCl₃) δ (ppm): 1.64-1.72 (m, 2H, —CH₂—), 1.88 (d, J=10.5 Hz, 1H, —CH₂—), 1.95-2.03 (m, 2H, —CH₂—), 2.29 (s, 4H, —CH—, —CH₃), 2.82 (br, 4H, —CH₂—), 2.95 (d, J=9.6 Hz, 1H, Ar—H₁), 3.40 (br, 4H, —CH₂—), 6.47-6.50 (m, 1H, Ar—H), 6.57 (s, 1H, Ar—H), 6.65 (d, J=8.4 Hz, 1H, Ar—H), 7.22 (d, J=8.1 Hz, 1H, Ar—H), 7.42-7.47 (m, 2H, Ar—H_{8,10}), 7.57-7.61 (m, 3H, Ar—H_{2,3,4}), 7.84 (d, J=8.4 Hz, 1H, Ar—H₇), 8.68 (d, J=7.8 Hz, 1H, Ar—H₁); ¹³C-NMR (300 MHz, CDCl₃) δ (ppm): 28.54, 46.34, 49.24, 50.49, 55.62, 61.83, 120.96, 124.27, 124.61, 125.04, 127.31, 128.45, 130.40, 131.54, 134.33, 135.15, 135.31, 136.55, 141.89, 149.81, 157.49, 194.38 (CO).

### Example 23

### 9-Chloro-6-(4-(1,4-dioxa-8-azaspiro[4,5]dec-8-yl)-11H-indeno[1, 2-c]quinolin-11-one (SJ-22)

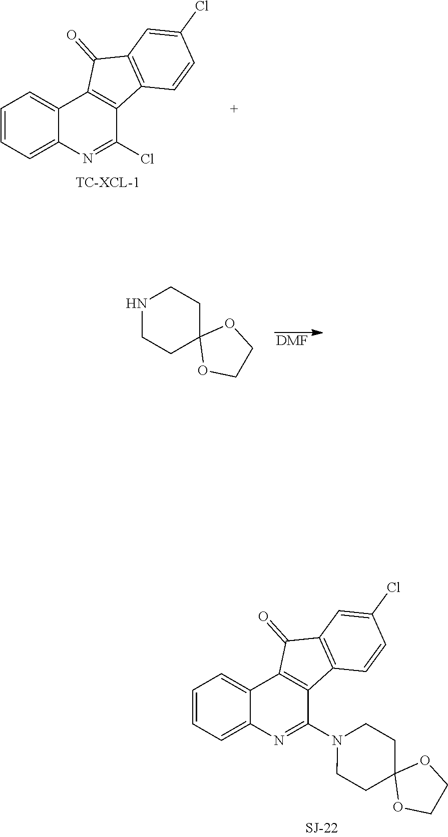

1,4-dioxa-8-azaspiro[4,5]dec-8-yl (1.29 mL, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-22 (0.23 g).

Mol. Wt.: 406.8616 (C₂₃H₁₉ClN₂O₃); R_{f}: 0.34 (Dichloromethane:Hexane=2:1); Yield: 56.0%; Mp.: 218-219° C. (EtOH); IR (KBr) cm⁻¹: 1718 (C═O); HRMS (ESI) m/z calcd for C₂₃H₁₉ClN₂O₃⁺[M]⁺: 406.1084. Found: [M+H]⁺=407.1154 (error<0.3%); ¹H-NMR (300 MHz, CDCl₃) δ (ppm): 1.98 (t, J=5.7 Hz, 4H, —CH₂—), 3.51 (br, 4H, —N—CH₂—), 4.04 (s, 4H, —O—CH₂—), 7.46 (td, J=8.7, 2.1 Hz, 2H, Ar—H_{8,10}), 7.57-7.62 (m, 3H, Ar—H_{2,3,4}), 7.83 (d, J=8.7 Hz, 1H, Ar—H₇), 8.69 (dd, J=8.4, 0.9 Hz, 1H, Ar—H₁); ¹³C-NMR (300 MHz, CDCl₃) δ (ppm): 35.00, 48.35, 64.66, 107.31, 120.98, 124.25, 125.06, 127.32, 128.41, 130.42, 131.65, 134.38, 135.09, 135.36, 136.57, 141.97, 149.74, 157.34, 194.43 (CO).

### Example 24

### 9-Chloro-6-(4-((piperazin-1-yl)(piperidin-1-yl)methanone)-11H-indeno[1,2-c]quinolin-11-one (SJ-23)

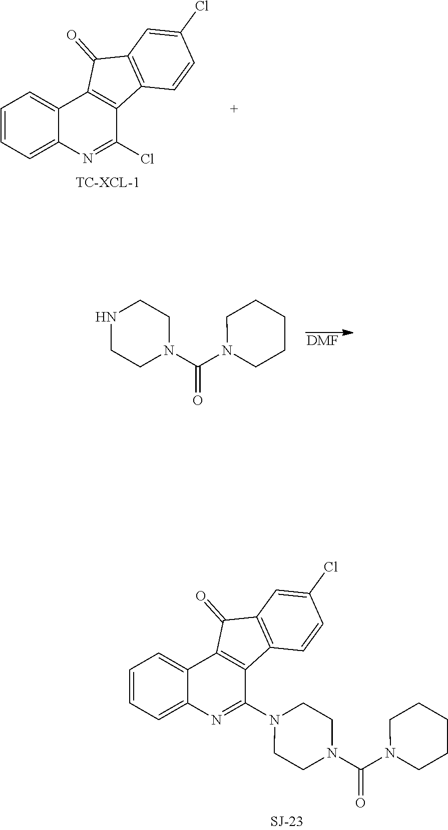

(piperazin-1-yl)(piperidin-1-yl)methanone (1.00 g, 10 mmole) was added to 10 mL DMF containing 0.3 g of 6,9-dichloro-11H-indeno[ 1,2-c]quinolin-11-one (TC-XCl-1) (1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-23 (0.22 g).

Mol. Wt.: 460.9553 (C₂₆H₂₅ClN₄O₂); R_{f}: 0.17 (Dichloromethane:Hexane=2:1); Yield: 46.9%; Mp.: 266-267° C. (EtOH); IR (KBr) cm⁻¹: 1718, 1647 (C═O stretch); HRMS (ESI) m/z calcd for C₂₃H₁₉ClN₂O₃⁺[M]⁺: 460.1666. Found: [M+H]⁺=461.1739 (error<0.3%); ¹H-NMR (300 MHz, CDCl₃) δ (ppm): 1.63 (s, 2H, —CH₂—), 3.28-3.47 (m, 16H, —CH₂—), 7.42-7.49 (m, 2H, Ar—H_{8,10}), 7.57-7.61 (m, 3H, Ar—H_{2,3,4}), 7.82 (d, J=8.4 Hz, 1H, Ar—H₇), 8.69 (d, J=7.8, 1H, Ar—H₁); ¹³C-NMR (300 MHz, CDCl₃) δ (ppm): 24.86, 25.97, 47.12, 47.99, 49.99, 121.16, 124.32, 124.47, 125.20, 127.65, 128.39, 130.58, 131.62, 134.49, 135.09, 135.54, 136.68, 141.56, 149.69, 157.36, 164.84 (CO), 194.20 (CO).

### Example 25

### 9-Chloro-6-(4-(3-(piperidin-4-yl)propyl)piperidin-1-yl)-11H-indeno[1,2-c]quinolin-11-one (SJ-24)

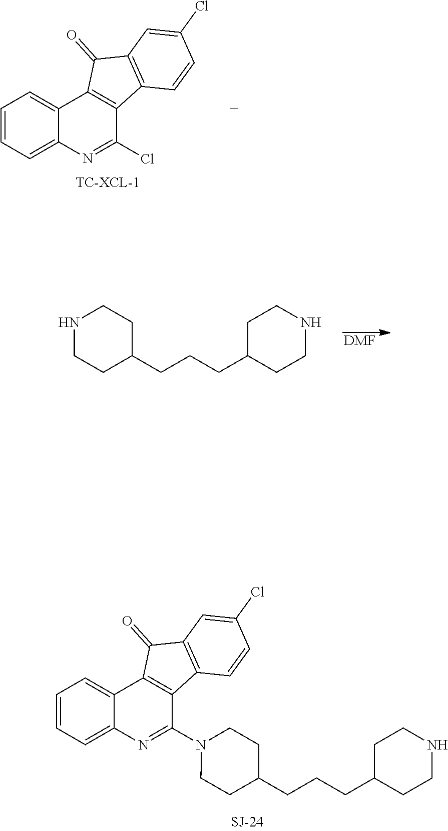

4-(3-(piperidin-4-yl)propyl)piperidine (2.10 g, 10 mmole) was added to 10 mL DMF containing 0.3 g of 6,9-dichloro-11H-indeno[1,2-c]quinolin-11-one (TC-XCl-1) (1 mmol). The mixture was heated while stirring at 150° C. for 4 hours and added pyridine (0.5 mL, 6.21 mmole) as the catalyst. Pour the mixture into ice water after the reaction is completed and filter to get the crude product. Recrystallized from hot EtOH to get compound SJ-24 (0.04 g).

Mol. Wt.: 474.0369 (C₂₉H₃₂ClN₃O); R_{f}: 0.41 (Dichloromethane:Hexane=2:1); Yield: 8.4%; Mp.: 149-151° C. (EtOH); IR (KBr) cm⁻¹: 1718 (C═O); HRMS (ESI) m/z calcd for C₂₉H₃₂ClN₃O [M]⁺: 473.2234. Found: [M+H]⁺=474.2318 (error<0.3%); ¹H-NMR (300 MHz, CDCl₃) δ (ppm): 1.07-1.46 (m, 12H, —CH₂—, —CH—), 1.66-1.91 (m, 4H, —CH₂—), 2.54-2.62 (m, 2H, N—CH₂₍ₐₓᵢₐₗ₎—), 2.90-2.94 (m, 2H, N—CH₂₍ₐₓᵢₐₗ₎—), 3.06 (d, J=12 Hz, 2H, N—CH_{2(equatorial)}—), 3.70 (d, J=12.3 Hz, 2H, N—CH_{2(equatorial)}—), 7.41-7.47 (m, 2H, Ar—H_{8,10}), 7.56-7.61 (m, 3H, Ar—H_{2,3,4}), 7.84 (d, J=8.4 Hz, 1H, Ar—H₇), 8.68 (d, J=8.1, 1H, Ar—H₁); ¹³C-NMR (300 MHz, CDCl₃) δ (ppm): 23.81, 32.58, 33.95, 35.88, 36.49, 37.04, 37.65, 47.07, 50.75, 120.89, 124.27, 124.44, 124.92, 127.15, 128.31, 130.32, 131.96, 134.33, 135.08, 135.23, 136.43, 142.12, 149.83, 158.22, 194.52 (CO).

### Example 26

### 6-(2-Hydroxyethylthio)-9-chloro-11H-indeno[1,2-c]quinolin-11-one (SJ-25)

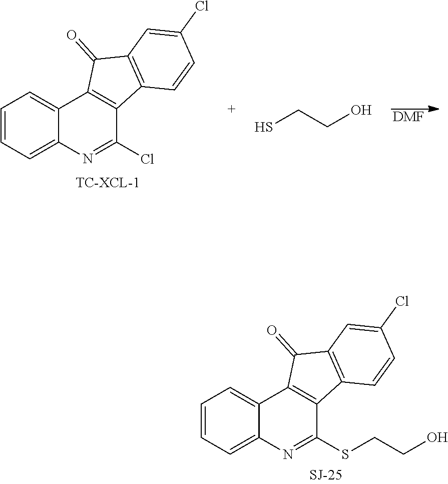

2-mercaptoethanol (0.78 g, 10 mmole) was added to 10 mL DMF containing 0.3 g of 6,9-dichloro-11H-indeno[1,2-c]quinolin-11-one (TC-XCl-1)(1 mmol). The mixture was heated while stirring at 150° C. for 4 hours and added potassium carbonate (0.27 g, 2 mmol) as catalyst. Pour the mixture into ice water after the reaction is completed and filter to get the crude product. Recrystallized from hot EtOH to get compound SJ-25 (0.33 g).

Mol. Wt.: 341.8114 (C₁₈H₁₂ClNO₂S); R_{f}: 0.23 (Dichloromethane:Hexane=2:1); Yield: 95.0%; Mp.: 169-170° C. (EtOH); IR (KBr) cm⁻¹: 1718 (C═O); HRMS (EI) m/z calcd for C₁₈H₁₂ClNO₂S⁺[M]⁺: 341.0277. Found: 341.0287 (error<0.3%); ¹H-NMR (300 MHz, CDCl₃) δ (ppm): 3.66 (t, J=5.4 Hz, 2H, —S—CH₂—), 4.11 (t, J=5.25 Hz, 2H, —CH₂—OH), 4.34 (br, 1H, OH), 7.47 (dd, J=8.4, 2.1 Hz, 1H, Ar—H₈), 7.53 (td, J=8.4, 1.5 Hz, 1H, Ar—H₂), 7.61 (d, J=2.1 Hz, 1H, Ar—H₁₀), 7.65 (td, J=8.4, 1.5 Hz, 1H, Ar—H₃), 7.87 (d, J=8.1 Hz, 1H, Ar—H₄), 7.92 (d, J=8.1 Hz, 1H, Ar—H₇), 8.72 (d, J=8.4 Hz, 1H, Ar—H₁); ¹³C-NMR (300 MHz, CDCl₃) δ (ppm): 34.05, 63.23, 121.54, 124.64, 125.36, 125.83, 127.90, 128.79, 131.14, 134.11, 134.61, 135.02, 136.08, 136.70, 140.76, 149.91, 155.11, 193.79 (CO).

### Example 27

### 6-Hydroxy-9-chloro-11H-indeno[1,2-c]quinolin-11-one (SJ-26)

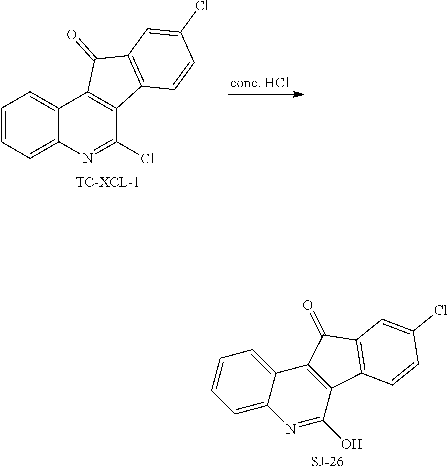 [SMILES: O=C1c2cc(Cl)ccc2-c2c(Cl)nc3ccccc3c21 ; O=C1c2cc(Cl)ccc2-c2c(O)nc3ccccc3c21]

Add 0.30 g of TC-XCl-1 (1.0 mmol) to 2 ml of 36% HCl and allow to reflux in DMF (20 mL) at 150° C. for 24 hours. Pour the mixture into ice water and incubate for 10 to 20 minutes. The precipitate collected at this stage is subjected to crystallization by ethanol to get the red compound SJ-26 (0.16 g, 57%).

Mol. Wt.: 281.6950 (C₁₆H₈ClNO₂); Yield: 57%; Mp.: 380° C.; HRMS (ESI) m/z calcd for C₁₆H₈NO₂Cl [M]⁺: 281.0244. Found: [M+H]⁺=282.0322, [M−H]⁻280.0178; [M−H]⁻280.0244; 1 H NMR (300 MHz, CDCl3) 7.29 (1H, td, J=7.5, 1.2 Hz, Ar—H2), 7.40 (1H, d, J=8.4 Hz, Ar—H9), 7.54-7.65 (3H, m, Ar—H3,7,8), 7.93 (1H, d, J=7.5 Hz, Ar—H4), 8.37 (1H, d, J=7.8 Hz, Ar—H1), 12.38 (br, 1H, —OH); ¹H NMR (400 MHz, DMSO-d6): δ ppm 7.30 (1H, t, J=7.6 Hz, Ar—H), 7.40 (1H, d, J=8.0 Hz, Ar—H), 7.56 (1H, t, J=7.6 Hz, Ar—H), 7.61 (1H, s, Ar—H), 7.63 (1H, d, J=7.6 Hz, Ar—H), 7.93 (1H, d, J=7.6 Hz, Ar—H), 8.37 (1H, d, J=8.4 Hz, Ar—H), 12.42 (1H, br, —OH); ¹³C NMR (100 MHz, DMSO-d6): 6 ppm 115.12, 116.39, 123.91, 124.41, 125.02, 131.62, 133.69, 134.33, 134.82, 135.68, 136.68, 140.78, 141.10, 159.20, 194.44.

### Example 28

### 6-Methoxy-9-chloro-11H-indeno[1,2-c]quinolin-11-one (SJ-27)

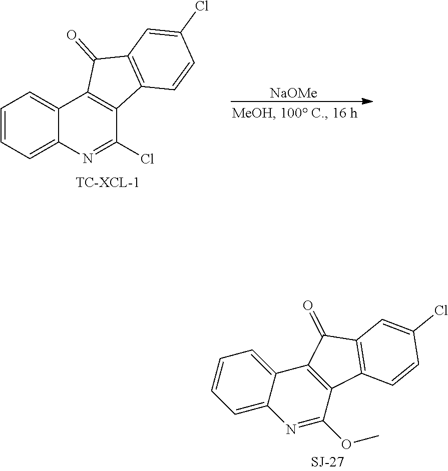 [SMILES: O=C1c2cc(Cl)ccc2-c2c(Cl)nc3ccccc3c21 ; COc1nc2ccccc2c2c1-c1ccc(Cl)cc1C2=O ; "NaOMe"]

Slowly added methanol containing 1.08 g of sodium methoxide (20 mmol) to TC-XCl-1/methanol (10 ml) and incubate for 10 minutes and allow to reflux in DMF (20 mL) at 90° C. for 10 hours (TLC monitored). After cooling, use a rotary evaporator to remove and filter the solvent and wash with alcohol and n-hexane to get compound SJ-27.

Mol. Wt.: 295.7220 (C₁₇H₁₀ClNO₂); R_{f}: 0.52 (CH₂Cl₂: n-hexane=1:1); Yield: 60%; Mp 259-261° C. (EtOH). ¹H NMR (400 MHz, CDCl₃): δ (ppm) 4.24 (3H, s, —OCH₃), 7.44 (1H, dd, J=8.0 Hz, 2.0 Hz, Ar—H), 7.47 (1H, td, J=7.6 Hz, 1.2 Hz, Ar—H), 7.59 (1H, d, J=2.0 Hz, Ar—H), 7.62 (1H, td, J=8.0 Hz, 1.6 Hz, Ar—H), 7.75 (1H, d, J=7.6 Hz, Ar—H), 7.85 (1H, d, J=8.4 Hz, Ar—H), 8.67 (1H, dd, J=8.0 Hz, 1.2 Hz, Ar—H). ¹³C NMR (100 MHz, CDCl₃): δ (ppm) 53.92, 120.66, 124.09, 124.86, 125.03, 126.68, 127.48, 129.08, 130.25, 134.31, 134.51, 135.16, 136.14, 140.25, 148.79, 158.13, 193.88 (C═O). HRMS (ESI) calcd for C₁₇H₁₀NO₂Cl [M]⁺295.0400. found [M+H]⁺296.0482.

Pharmacological Activity Assay

In pharmacological tests, compounds synthesized chemically including TC-XCl-1 and SJ-1 through SJ-27 (a total of 28 drugs) are subjected to the following pharmacological activity tests: (1) SRB assay, (2) Topoisomerase I Drug Screening Test, and (3) cytotoxicity assays conducted by NCI on the 16 screened compounds in 55 to 60 cancer cell lines.

### Example 29

### Sulforhodamine B (SRB) Assay

Add 5% Fetal Bovine Serum (FBS) to 96-well plate and fix the cells with 10% TCA after 24 hours of incubation and add the compound (T₀). Add DMSO and culture for another 48 hours and the cells are fixed in 0.4% (W/V), 10% TCA and SRB, and stained with 1% acetic acid. Excess SRB is washed off by 1% acetic acid and cells stained with SRB are dissolved in 10 mmol/L Trizma base. The absorbance is measured at 515 nm. The treatment time is 0 (T₀), control growth (C) and the increase of the compound (Tx) in the cells are shown below and are used to calculate the increase of the concentration of the compound in percentage. Growth inhibition (GI %) is calculated using the following formula 100−[(T_{X}−T₀)/(C−T₀)]×100. The 50% growth inhibition concentration (IC₅₀) is used to confirm the concentration of compound and thus results in 50% reduction of the treated cells which control the amount of total protein.

This part of experimental results are SRB assays measuring cytotoxicity of the 26 synthesized compounds against human PC-3 cells and are summarized in Table 1 and 2.

TABLE 1
The SRB assay results (I)

| Compound | IC₅₀ (μM) |
|---|---|
| TC-XCl-1 | >15 |
| SJ-2 | 4.34 |
| SJ-8 | >10 |
| SJ-25 | >10 |

TABLE 2
The SRB assay results (II)

| | | GI % | Tox- |
|---|---|---|---|
| | | (10 | icity |
| Compd. | —R | μM) | (%) |
| SJ-1 | 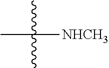 [SMILES: *NC] | 100 | 52.8 |
| SJ-3 | 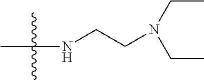 [SMILES: *NCCN(CC)CC] | 100 | 55.6 |
| SJ-4 | 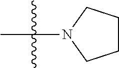 [SMILES: *N1CCCC1] | 100 | 47.2 |
| SJ-5 | 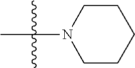 [SMILES: *N1CCCCC1] | 100 | 22.2 |
| SJ-6 | 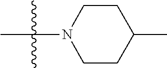 [SMILES: *N1CCC(C)CC1] | 89.81 | 0 |
| SJ-7 | 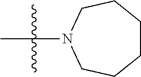 [SMILES: *N1CCCCCC1] | 100 | 41.7 |
| SJ-9 | 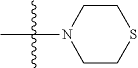 [SMILES: *N1CCSCC1] | 100 | 33.3 |
| SJ-10 | 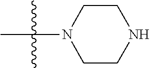 [SMILES: *N1CCNCC1] | 100 | 47.2 |
| SJ-11 | 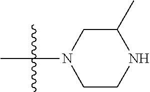 [SMILES: *N1CCNC(C)C1] | 100 | 64.9 |
| SJ-12 | 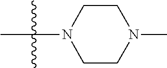 [SMILES: *N1CCN(C)CC1] | 39.4 | 0 |
| SJ-13 | 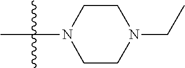 [SMILES: *N1CCN(CC)CC1] | 100 | 29.7 |
| SJ-14 | 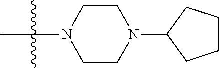 [SMILES: *N1CCN(C2CCCC2)CC1] | 96.26 | 0 |
| SJ-15 | 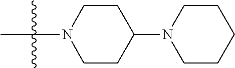 [SMILES: *N1CCC(N2CCCCC2)CC1] | 98.83 | 0 |
| SJ-16 | 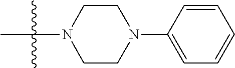 [SMILES: *N1CCN(c2ccccc2)CC1] | 65.91 | 0 |
| SJ-17 | 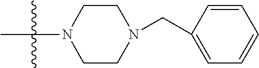 [SMILES: *N1CCN(Cc2ccccc2)CC1] | 73.53 | 0 |
| SJ-18 | 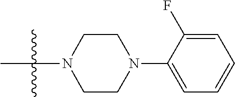 [SMILES: *N1CCN(c2ccccc2F)CC1] | 85.48 | 0 |
| SJ-19 | 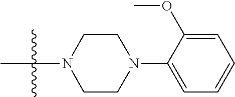 [SMILES: *N1CCN(c2ccccc2OC)CC1] | 100 | 8.5 |
| SJ-20 | 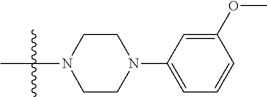 [SMILES: *N1CCN(c2cccc(OC)c2)CC1] | 98.05 | 0 |
| SJ-21 | 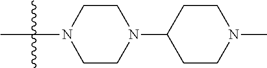 [SMILES: *N1CCN(C2CCN(C)CC2)CC1] | 100 | 66 |
| SJ-22 | 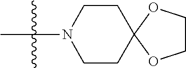 [SMILES: *N1CCC2(CC1)OCCO2] | 100 | 42.6 |
| SJ-23 | 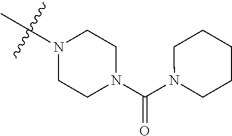 [SMILES: *N1CCN(C(=O)N2CCCCC2)CC1] | 100 | 43 |
| SJ-24 | 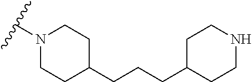 [SMILES: *N1CCC(CCCC2CCNCC2)CC1] | 100 | 63.8 |

### Example 30

### Topoisomerase I Drug Screening

Four compounds are selected (SJ-2, SJ-3, SJ-11 and SJ-24) and are used in SRB assays for examination of their cytotoxicity effects against PC-3 cells based on the following principles.

TopoGEN Topoisomerase I Drug Screening Kit comprises necessary reagents for examining whether the compound has inhibition effects on topoisomerase I (Topo I). This kit can detect compounds utilizing two different mechanisms for inhibition of Top I, the first type inhibits enzyme activity and is called catalytic inhibitory compounds (CICs) and the second type stimulates stable formation of the drug-Top I-DNA cleavage complex and is referred to interfacial poisons (IFPs). Though CICs affect various levels of the enzyme, such as inhibition of the interaction between Topo I and DNA, which is non-specific, but high concentration of salts or polyamines are strong CICs. IFPs, on the other hand, inhibits ligation between nicked DNA. Camptothecin (CPT) and its derivatives are IFPs and are used as positive control in this kit. Under normal conditions, the mechanism of Topo I allows Topo I to enter a breakage/resealing DNA cycle. The cleavage stage during the process is very short and cannot be detected, whereas IFPs, such as integration of Camptothecin into the cleavage complex which leads to interruption of the resealing step and consequently leave the DNA in the nicked (ss break) form and linked to Topo I at the 5′ end of the nick on the DNA by covalent bonds while 3′ end is free. This kit utilizes circular supercoiled plasmid DNA (pHOT1) as the matrix. Topo I relaxes DNA and makes the DNA circular and prevents formation of nicked DNA. Following addition of the drug CPT, proteinase K will denature Topo I and produce nicked (nicked and unsealed) DNA, and this step is necessary for determination of unligated plasmid. If the tested compounds are CICs which can effectively inhibit enzyme activity, no relaxed DNA will be detected in the experiment.

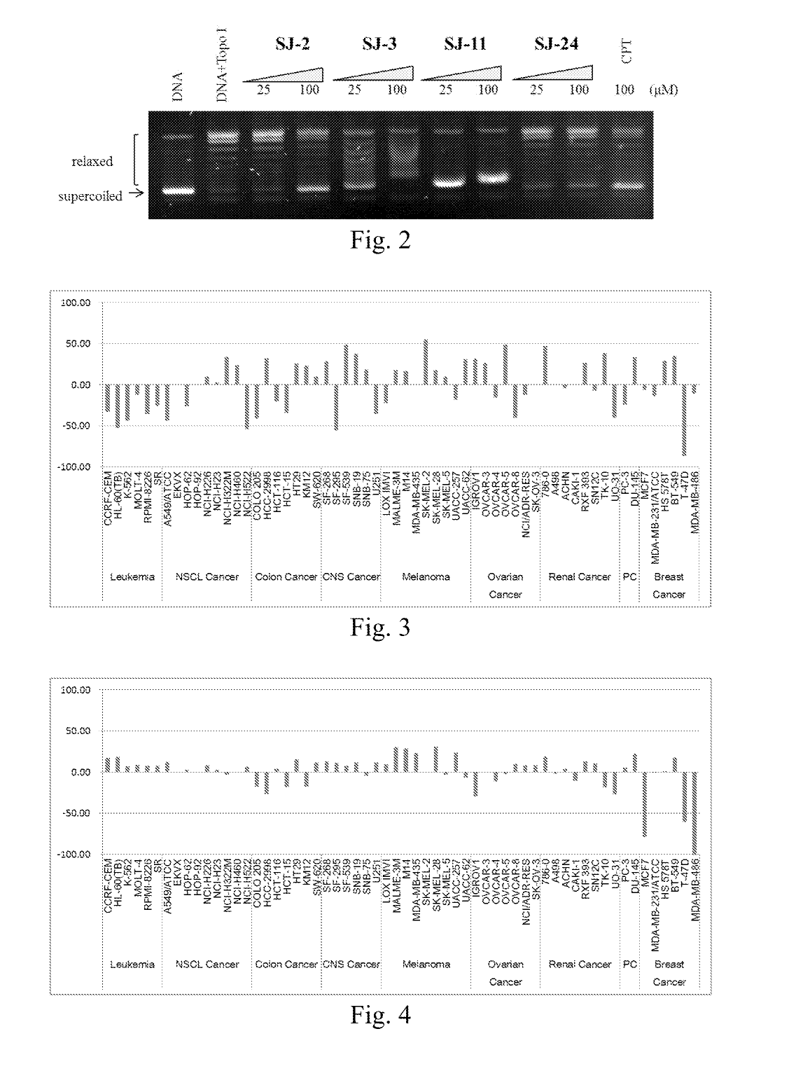

Based on the results obtained from SRB assays, preliminary tests were conducted to examine the inhibition effects of the four compounds with better drug efficacy, SJ-2, SJ-3, SJ-11 and SJ-24, at the concentrations of 25 μM and 100 μM and the control group camptothecin (CPT) at 100 μM, in duplicate. The results indicate that SJ-11 is more effective than CPT in inhibition of topoisomerase I at the concentration of 25 μM (FIG. 2). The solubility of the four compounds in DMSO is poor, SJ-2<SJ-24<SJ-3<SJ-11, and therefore it is estimated and this is the reason why the three compounds which have previously proven to be effective against PC-3 cell line by SRB assays are ineffective in this experiment.

### Example 31

### National Cancer Institute Cancer Cell Cytotoxicity Assay

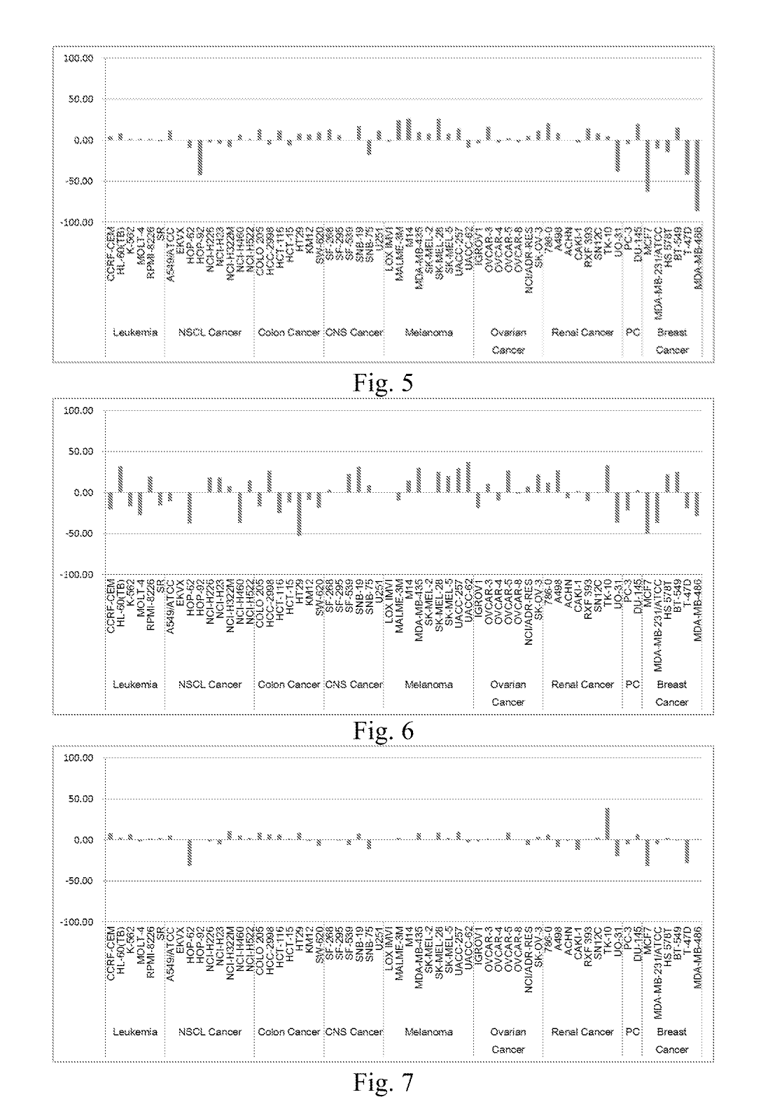

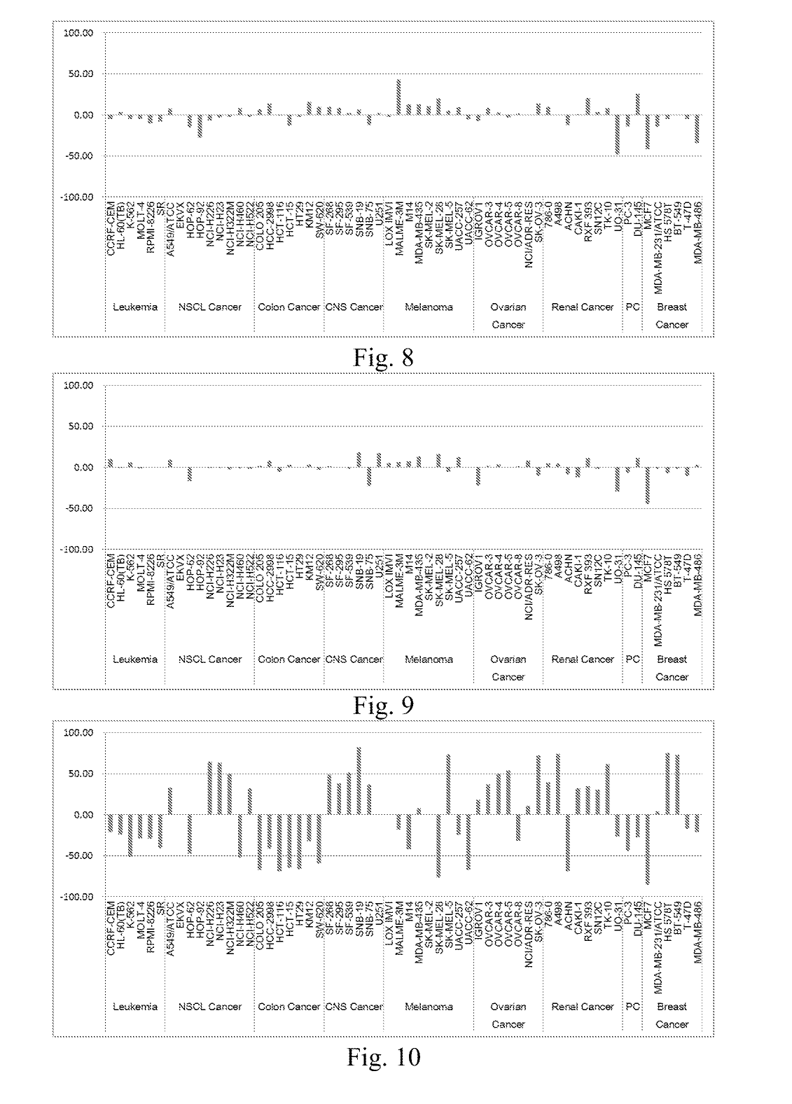

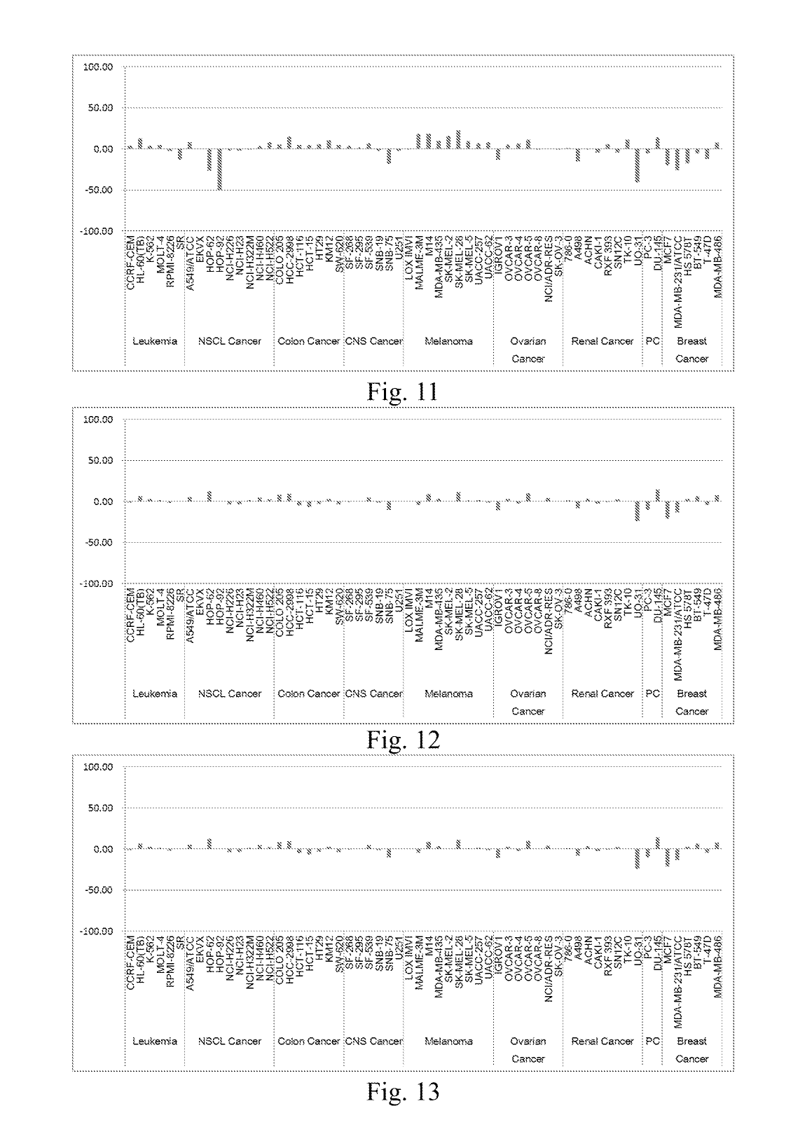

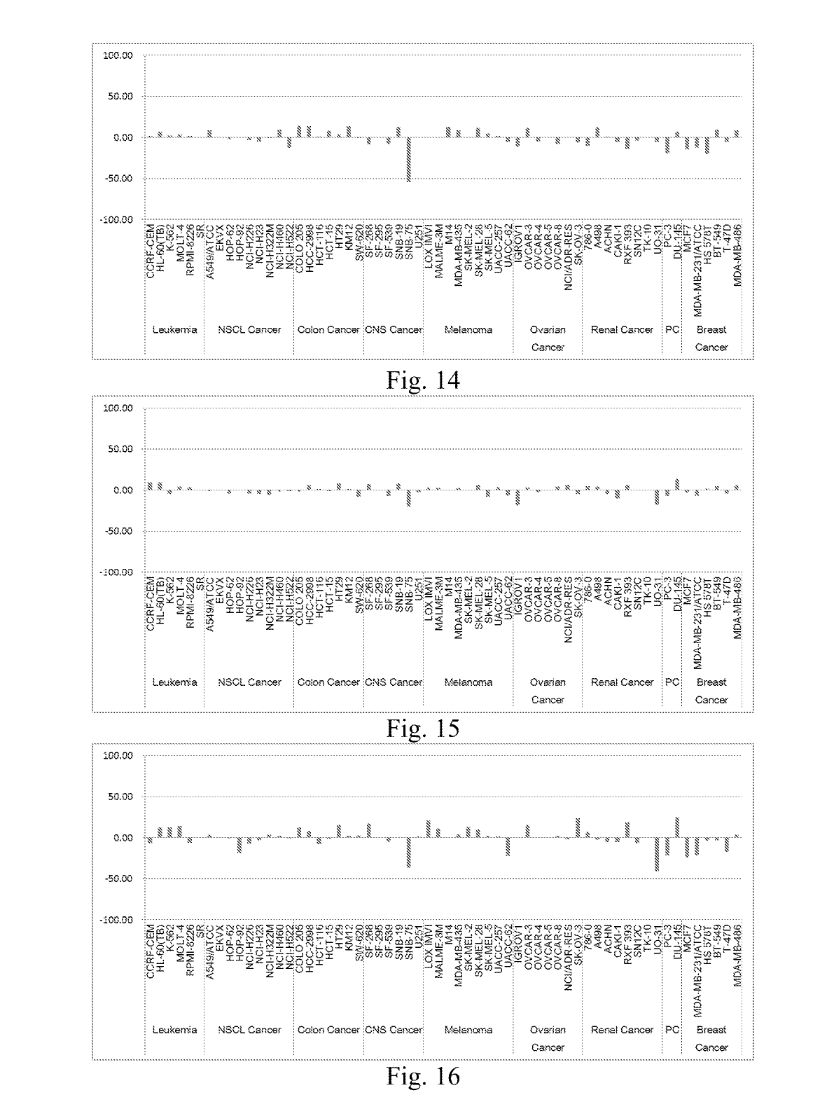

The test results shown in this section are the compound cytotoxicities in vitro against cancer cell lines National Cancer Institute (NCI)'s anticancer drug screen and 16 (TC-XLC-1, SJ-1, SJ-2, SJ-3, SJ-6, SJ-8, SJ-9, SJ-10, SJ-12, SJ-14, SJ-16, SJ-20, SJ-23, SJ-25, SJ-26 and SJ-27) were selected from the original 28 compounds screened. In the first stage, cytotoxicity of the 16 compounds at the concentration of 10 μM was conducted on 55 to 60 cell lines and SRB assay was performed after 48 hours of incubation. The results are shown in FIGS. 3 to 16 and Tables 3 to 7 and are represented by growth percentage. The mean of the growth percentage of all tested cell lines in a group is deducted from the obtained results and is used for determination of the cytotoxicity effect of that particular compound in the tested cancer cell line.

Two compounds SJ-3 and SJ-10 were found to have potential cytotoxicity and were subjected to the cytotoxicity tests at 5 different concentrations in the second stage. From the results, compounds SJ-3 (NSC 772864) and SJ-10 (NSC 772862) were selected by NCI for a five-dose level screening test and the mean growth percentage of the 60 cancer cell lines for the two compounds are 62.4 and −14.37, respectively. Preliminary analysis of the test results of the 16 compounds selected in the first stage suggests that substitution of the replacement functional group at site 6 of TC-XCl-1 induces more significant selective inhibition effect in breast cancer MCF-7 cell line. A summary of the NCI data provides information on the inhibition effect of various compound in different cancer cell lines (FIGS. 3 to 16 and Tables 3 to 8). For example, compound TC-XCl-1 can inhibit the growth of 9 cancer cell lines including leukemia and breast cancer cells, whereas compounds SJ-1 and SJ-2 are more effective in breast cancer cell line and compound SJ-10 show preferable inhibition effects in leukemia and colon cancer cells. Table 9 indicates the GI₅₀, TGI and LC₅₀ of the NCI data of TC-XCl, SJ-3 and SJ-10 NCI.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

TABLE 3
TC-XCl-1, SJ-1 and SJ-2 data collected from NCI

| | | TC-XCL-1 | SJ-1 | SJ-2 |
|---|---|---|---|---|
| Compound No. | | NSC | NSC | NSC |
| NCI No. | | 763969 | 772856 | 771781 |
| Leukemia | CCRF-CEM | 64.86 | 101.50 | 92.13 |
| | HL-60 (TB) | 102.00 | 102.75 | 95.49 |
| | K-562 | 102.16 | 90.99 | 88.81 |
| | MOLT-4 | 70.83 | 93.26 | 88.92 |
| | RPMI-8226 | 92.83 | 91.82 | 88.05 |
| | SR | 65.85 | 91.67 | 86.12 |
| Non-Small Cell | A549/ATCC | 108.98 | 96.83 | 98.85 |
| Lung Cancer | EKVX | 79.27 | N.T. | N.T. |
| | HOP-62 | 94.89 | 87.10 | 78.02 |
| | HOP-92 | 71.94 | N.T. | 44.26 |
| | NCI-H226 | 91.86 | 92.19 | 84.79 |
| | NCI-H23 | 79.30 | 86.83 | 82.62 |
| | NCI-H322M | 95.38 | 80.85 | 78.83 |
| | NCI-H460 | 98.68 | 84.38 | 94.17 |
| | NCI-H522 | 89.37 | 90.50 | 88.97 |
| Colon Cancer | COLO 205 | 99.18 | 66.47 | 100.97 |
| | HCC-2998 | 98.52 | 57.51 | 81.50 |
| | HCT-116 | 105.87 | 88.31 | 98.57 |
| | HCT-15 | 114.88 | 66.20 | 80.49 |
| | HT29 | 104.27 | 99.44 | 94.89 |
| | KM12 | 111.92 | 66.82 | 94.44 |
| | SW-620 | 98.29 | 96.25 | 96.35 |
| CNS Cancer | SF-268 | 117.27 | 97.27 | 100.26 |
| | SF-295 | 92.80 | 95.71 | 93.22 |
| | SF-539 | 110.08 | 92.08 | 87.80 |
| | SNB-19 | 101.30 | 95.95 | 104.19 |
| | SNB-75 | 82.91 | 79.70 | 69.12 |
| | U251 | 100.63 | 96.03 | 98.98 |
| Melanoma | LOX IMVI | 84.23 | 93.40 | 85.45 |
| | MALME-3M | 102.06 | 114.83 | 111.50 |
| | M14 | 109.79 | 112.68 | 113.63 |
| | MDA-MB-435 | 100.46 | 107.24 | 97.33 |
| | SK-MEL-2 | 117.11 | N.T. | 95.09 |
| | SK-MEL-28 | 90.08 | 114.89 | 113.43 |
| | SK-MEL-5 | 86.30 | 80.96 | 94.89 |
| | UACC-257 | 127.99 | 107.69 | 101.11 |
| | UACC-62 | 94.45 | 77.41 | 78.16 |
| Ovarian Cancer | IGROV1 | 92.82 | 54.41 | 83.38 |
| | OVCAR-3 | 119.93 | 84.91 | 103.48 |
| | OVCAR-4 | 94.13 | 72.83 | 84.28 |
| | OVCAR-5 | 137.00 | 81.54 | 89.14 |
| | OVCAR-8 | 101.08 | 94.33 | 84.55 |
| | NCI/ADR-RES | 96.78 | 92.22 | 92.57 |
| | SK-OV-3 | N.T. | 92.62 | 99.12 |
| Renal Cancer | 786-0 | 112.40 | 103.34 | 108.01 |
| | A498 | N.T. | 82.03 | 96.00 |
| | ACHN | 111.67 | 88.46 | 87.55 |
| | CAKI-1 | 75.67 | 73.84 | 84.65 |
| | RXF 393 | 112.80 | 97.15 | 101.32 |
| | SN12C | 88.03 | 94.82 | 95.38 |
| | TK-10 | 138.45 | 65.31 | 91.84 |
| | UO-31 | 80.62 | 56.91 | 48.63 |
| Prostate Cancer | PC-3 | 92.73 | 89.44 | 82.55 |
| | DU-145 | 113.91 | 106.88 | 106.50 |
| Breast Cancer | MCF7 | 74.25 | 5.24 | 23.99 |
| | MDA-MB-231/ | 87.66 | 83.87 | 76.68 |
| | ATCC | | | |
| | HS 578T | 91.01 | 85.28 | 72.84 |
| | BT-549 | 119.62 | 101.88 | 102.55 |
| | T-47D | 86.97 | 23.53 | 44.88 |
| | MDA-MB-468 | 107.92 | -31.61 | 0.60 |
| Mean | | 98.17 | 84.15 | 87.15 |
| Delta | | 33.31 | 115.76 | 86.55 |
| Range | | 73.59 | 146.5 | 113.03 |

TABLE 4
SJ-3, SJ-6 and SJ-8 data collected from NCI

| | | SJ-3 | SJ-6 | SJ-8 |
|---|---|---|---|---|
| Compound No. | | NSC | NSC | NSC |
| NCI No. | | 772864 | 772860 | 771782 |
| Leukemia | CCRF-CEM | 42.00 | 104.33 | 88.85 |
| | HL-60 (TB) | 94.33 | 98.84 | 97.09 |
| | K-562 | 45.29 | 103.57 | 89.01 |
| | MOLT-4 | 34.63 | 94.41 | 88.74 |
| | RPMI- 8226 | 81.94 | 97.99 | 83.40 |
| | SR | 46.92 | 98.51 | 85.94 |
| Non-Small Cell | A549/ATCC | 51.79 | 101.69 | 101.47 |
| Lung Cancer | EKVX | N.T. | N.T. | N.T. |
| | HOP-62 | 24.86 | 64.18 | 78.71 |
| | HOP-92 | N.T. | N.T. | 66.08 |
| | NCI-H226 | 80.86 | 94.59 | 87.21 |
| | NCI-H23 | 80.63 | 90.42 | 90.40 |
| | NCI-H322M | 69.83 | 107.08 | 91.28 |
| | NCI-H460 | 25.15 | 101.21 | 102.14 |
| | NCI-H522 | 77.39 | 98.45 | 91.11 |
| Colon Cancer | COLO 205 | 45.69 | 104.79 | 100.71 |
| | HCC-2998 | 88.65 | 102.76 | 107.23 |
| | HCT-116 | 36.96 | 102.51 | 93.64 |
| | HCT-15 | 50.38 | 97.78 | 80.29 |
| | HT29 | 9.75 | 104.84 | 91.34 |
| | KM12 | 53.42 | 94.80 | 109.99 |
| | SW-620 | 43.72 | 88.89 | 103.71 |
| CNS Cancer | SF-268 | 65.88 | 96.49 | 103.58 |
| | SF-295 | N.T. | 96.18 | 102.43 |
| | SF-539 | 85.04 | 90.22 | 96.24 |
| | SNB-19 | 94.02 | 103.87 | 100.01 |
| | SNB-75 | 71.52 | 84.42 | 81.55 |
| | U251 | N.T. | N.T. | 95.72 |
| Melanoma | LOX IMVI | N.T. | N.T. | 91.20 |
| | MALME-3M | 52.55 | 98.31 | 136.93 |
| | M14 | 76.44 | 96.60 | 106.99 |
| | MDA-MB-435 | 92.72 | 104.53 | 106.62 |
| | SK-MEL-2 | N.T. | N.T. | 104.29 |
| | SK-MEL-28 | 87.40 | 105.21 | 113.81 |
| | 5K-MEL-5 | 82.51 | 98.52 | 99.19 |
| | UACC-257 | 92.18 | 106.47 | 103.31 |
| | UACC-62 | 99.56 | 93.36 | 88.33 |
| Ovarian Cancer | IGROV1 | 43.43 | 94.36 | 86.47 |
| | OVCAR-3 | 73.13 | 97.59 | 102.34 |
| | OVCAR-4 | 52.50 | 97.11 | 96.67 |
| | OVCAR-5 | 89.34 | 104.92 | 90.30 |
| | OVCAR-8 | 61.36 | 96.38 | 95.25 |
| | NCI/ADR-RES | 69.55 | 90.28 | 94.32 |
| | SK-OV-3 | 84.20 | 99.78 | 107.26 |
| Renal Cancer | 786-0 | 74.13 | 102.73 | 103.71 |
| | A498 | 88.98 | 87.55 | 93.88 |
| | ACHN | 55.86 | 94.75 | 81.66 |
| | CAKI-1 | 64.35 | 83.89 | 94.99 |
| | RXF 393 | 51.90 | 97.51 | 114.61 |
| | SN12C | 61.60 | 99.08 | 96.95 |
| | TK-10 | 95.55 | 134.79 | 101.98 |
| | UO-31 | 25.86 | 76.25 | 45.74 |
| Prostate Cancer | PC-3 | 41.01 | 90.64 | 79.64 |
| | DU-145 | 65.48 | 103.47 | 119.02 |
| Breast Cancer | MCF7 | 12.70 | 64.65 | 52.31 |
| | MDA-MB-231/ | 25.16 | 90.96 | 79.28 |
| | ATCC | | | |
| | HS 578T | 84.70 | 98.38 | 88.93 |
| | BT-549 | 87.48 | 95.52 | 93.95 |
| | T-47D | 43.28 | 67.68 | 88.81 |
| | MDA-MB-468 | 33.81 | 97.12 | 59.47 |
| Mean | | 62.4 | 96.2 | 93.66 |
| Delta | | 52.65 | 32.02 | 47.92 |
| Range | | 89.81 | 70.61 | 91.19 |

TABLE 5
SJ-9, SJ-10 and SJ-12 data collected from NCI

| | | SJ-9 | SJ-10 | SJ-12 |
|---|---|---|---|---|
| Compound No. | | NSC | NSC | NSC |
| NCI No. | | 772857 | 772862 | 771783 |
| Leukemia | CCRF-CEM | 105.04 | -35.45 | 92.44 |
| | HL-60 (TB) | 94.50 | -38.36 | 101.15 |
| | K-562 | 100.89 | -65.71 | 92.13 |
| | MOLT-4 | 93.29 | -43.22 | 92.90 |
| | RPMI-8226 | 95.20 | -43.57 | 85.92 |
| | SR | 99.53 | -55.19 | 75.13 |
| Non-Small Cell | A549/ATCC | 104.35 | 18.70 | 96.43 |
| Lung Cancer | EKVX | N.T. | N.T. | N.T. |
| | HOP-62 | 78.17 | -61.80 | 61.42 |
| | HOP-92 | N.T. | N.T. | 38.50 |
| | NCI-H226 | 94.22 | 49.94 | 86.17 |
| | NCI-H23 | 94.33 | 49.19 | 86.27 |
| | NCI-H322M | 92.29 | 35.89 | 87.41 |
| | NCI-H460 | 93.32 | -66.36 | 91.83 |
| | NCI-H522 | 92.76 | 18.08 | 96.62 |
| Colon Cancer | COLO 205 | 96.30 | -81.49 | 93.65 |
| | HCC-2998 | 103.16 | -55.83 | 103.21 |
| | HCT-116 | 89.08 | -83.19 | 92.78 |
| | HCT-15 | 97.88 | -78.92 | 92.83 |
| | HT29 | 95.52 | -80.89 | 93.76 |
| | KM12 | 97.97 | -47.09 | 99.06 |
| | SW-620 | 91.88 | -73.67 | 92.76 |
| CNS Cancer | SF-268 | 96.13 | 34.33 | 92.35 |
| | SF-295 | 93.33 | 23.75 | 90.01 |
| | SF-539 | 92.93 | 36.91 | 95.23 |
| | SNB-19 | 113.43 | 67.25 | 85.41 |
| | SNB-75 | 72.13 | 22.34 | 70.00 |
| | U251 | 111.96 | N.T. | 85.86 |
| Melanoma | LOX IMVI | 100.04 | N.T. | 87.17 |
| | MALME-3M | 101.05 | -32.28 | 106.59 |
| | M14 | 101.69 | -56.24 | 106.31 |
| | MDA-MB-435 | 107.70 | -6.57 | 98.43 |
| | SK-MEL-2 | N.T. | N.T. | 104.36 |
| | SK-MEL-28 | 110.84 | -91.38 | 110.81 |
| | SK-MEL-5 | 89.52 | 59.18 | 97.67 |
| | UACC-257 | 107.44 | -39.24 | 95.34 |
| | UACC-62 | 94.90 | -81.71 | 96.67 |
| Ovarian Cancer | IGROV1 | 72.45 | 3.23 | 75.03 |
| | OVCAR-3 | 96.58 | 22.86 | 93.41 |
| | OVCAR-4 | 98.19 | 35.59 | 94.70 |
| | OVCAR-5 | 94.37 | 39.14 | 99.31 |
| | OVCAR-8 | 96.11 | -46.09 | 87.51 |
| | NCI/ADR-RES | 102.85 | -4.00 | 88.19 |
| | SK-OV-3 | 84.60 | 57.81 | 87.98 |
| Renal Cancer | 786-0 | 99.53 | 25.31 | 89.51 |
| | A498 | 99.15 | 59.79 | 72.81 |
| | ACHN | 86.42 | -83.29 | 88.10 |
| | CAKI-1 | 82.68 | 17.84 | 83.81 |
| | RXF 393 | 106.09 | 20.51 | 94.00 |
| | SN12C | 93.10 | 15.89 | 83.57 |
| | TK-10 | 121.79 | 47.07 | 99.56 |
| | UO-31 | 65.33 | -40.61 | 47.60 |
| Prostate Cancer | PC-3 | 87.77 | -58.51 | 82.93 |
| | DU-145 | 106.04 | -41.83 | 102.37 |
| Breast Cancer | MCF7 | 49.61 | -100.00 | 68.30 |
| | MDA-MB-231/ | 93.08 | -10.45 | 62.53 |
| | ATCC | | | |
| | HS 578T | 87.68 | 60.88 | 70.47 |
| | BT-549 | 92.91 | 58.35 | 82.89 |
| | T-47D | 83.86 | -31.54 | 76.27 |
| | MDA-MB-468 | 97.71 | -35.66 | 95.71 |
| Mean | | 94.75 | -14.37 | 88.15 |
| Delta | | 45.14 | 85.63 | 49.65 |
| Range | | 72.18 | 167.25 | 72.31 |

TABLE 6
SJ-14, SJ-16 and SJ-20 data collected from NCI

| | | SJ-14 | SJ-16 | SJ-20 |
|---|---|---|---|---|
| Compound No. | | NSC | NSC | NSC |
| NCI No. | | 772859 | 7772861 | 772863 |
| Leukemia | CCRF-CEM | 95.17 | 93.52 | 93.78 |
| | HL-60 (TB) | 103.04 | 105.45 | 99.09 |
| | K-562 | 99.26 | 95.17 | 94.25 |
| | MOLT-4 | 98.39 | 98.13 | 95.46 |
| | RPMI-8226 | 94.89 | 95.33 | 93.63 |
| | SR | 97.09 | 95.93 | 95.05 |
| Non-Small Cell | A549/ATCC | 101.56 | 90.89 | 101.22 |
| Lung Cancer | EKVX | N.T. | N.T. | N.T. |
| | HOP-62 | 108.83 | N.T. | 90.22 |
| | HOP-92 | N.T. | N.T. | N.T. |
| | NCI-H226 | 92.80 | 79.43 | 89.19 |
| | NCI-H23 | 92.80 | 83.34 | 87.22 |
| | NCI-H322M | 98.50 | 98.63 | 91.93 |
| | NCI-H460 | 101.46 | 97.04 | 101.76 |
| | NCI-H522 | 98.72 | 80.10 | 79.75 |
| Colon Cancer | COLO 205 | 104.54 | 102.19 | 106.63 |
| | HCC-2998 | 106.04 | 106.23 | 105.79 |
| | HCT-116 | 91.34 | 78.18 | 93.02 |
| | HCT-15 | 89.64 | 80.71 | 100.39 |
| | HT29 | 93.22 | 82.40 | 95.93 |
| | KM12 | 99.33 | 98.34 | 105.95 |
| | SW-620 | 93.06 | 93.14 | 91.16 |
| CNS Cancer | SF-268 | 96.39 | 85.45 | 83.89 |
| | SF-295 | 96.87 | 60.91 | 90.05 |
| | SF-539 | 101.34 | 83.17 | 83.97 |
| | SNB-19 | 95.06 | 88.40 | 105.27 |
| | SNB-75 | 86.36 | 33.20 | 37.76 |
| | U251 | N.T. | N.T. | N.T. |
| Melanoma | LOX IMVI | N.T. | N.T. | N.T. |
| | MALME-3M | 92.48 | 100.05 | 92.79 |
| | M14 | 105.34 | 97.52 | 105.07 |
| | MDA-MB-435 | 99.93 | 99.55 | 100.75 |
| | SK-MEL-2 | N.T. | N.T. | N.T. |
| | SK-MEL-28 | 107.58 | 104.82 | 103.93 |
| | 5K-MEL-5 | 97.48 | 99.23 | 96.87 |
| | UACC-257 | 98.37 | 98.40 | 93.79 |
| | UACC-62 | 95.06 | 87.17 | 86.44 |
| Ovarian Cancer | IGROV1 | 85.84 | 65.13 | 80.87 |
| | OVCAR-3 | 99.54 | 98.07 | 104.27 |
| | OVCAR-4 | 94.20 | 90.32 | 87.08 |
| | OVCAR-5 | 106.41 | 88.56 | 98.22 |
| | OVCAR- 8 | 96.55 | 75.82 | 83.98 |
| | NCI/ADR-RPS | 100.39 | 97.88 | 92.82 |
| | SK-OV-3 | 96.85 | 78.58 | 86.02 |
| Renal Cancer | 786-0 | 97.90 | 81.41 | 81.67 |
| | A498 | 88.64 | 97.48 | 104.53 |
| | ACHN | 100.03 | 68.58 | 93.51 |
| | CAKI-1 | 93.83 | 80.23 | 86.59 |
| | RXF 393 | 95.60 | 72.95 | 78.38 |
| | SN12C | 99.08 | 87.90 | 88.17 |
| | TK-10 | N.T. | 115.61 | 133.34 |
| | UO-31 | 72.57 | 65.02 | 86.64 |
| Prostate Cancer | PC-3 | 86.19 | 79.79 | 72.83 |
| | DU-145 | 110.73 | 102.96 | 98.98 |
| Breast Cancer | MCF7 | 75.18 | 53.31 | 77.46 |
| | MDA-MB-231/ | 82.96 | 73.80 | 79.81 |
| | ATCC | | | |
| | HS 578T | 99.40 | N.T. | 71.75 |
| | BT-549 | 102.74 | 89.83 | 101.57 |
| | T-47D | 92.20 | 75.93 | 86.68 |
| | MDA-MB-468 | 103.99 | 95.37 | 101.04 |
| Mean | | 96.53 | 87.29 | 92.15 |
| Delta | | 23.96 | 54.09 | 54.39 |
| Range | | 38.16 | 82.41 | 95.58 |

TABLE 7
SJ-23 data collected from NCI

| | | SJ-23 |
|---|---|---|
| Compound No. | | NSC |
| NCI No. | | 772858 |
| Leukemia | CCRF-CEM | 109.87 |
| | HL-60 (TB) | 109.78 |
| | K-562 | 95.70 |
| | MOLT-4 | 104.77 |
| | RPMI-8226 | 104.31 |
| | SR | 105.15 |
| Non-Small Cell | A549/ATCC | 99.12 |
| Lung Cancer | EKVX | N.T. |
| | HOP-62 | 96.39 |
| | HOP-92 | N.T. |
| | NCI-H226 | 96.06 |
| | NCI-H23 | 95.41 |
| | NCI-H322M | 94.59 |
| | NCI-H460 | 98.85 |
| | NCI-H522 | 99.28 |
| Colon Cancer | COLO 205 | 98.52 |
| | HCC-2998 | 106.23 |
| | HCT-116 | 101.67 |
| | HCT-15 | 99.35 |
| | HT29 | 108.65 |
| | KM12 | 101.58 |
| | SW-620 | 92.51 |
| CNS Cancer | SF-268 | 107.51 |
| | SF-295 | 97.83 |
| | SF-539 | 92.98 |
| | SNB-19 | 108.66 |
| | SNB-75 | 80.84 |
| | U251 | 98.15 |
| Melanoma | LOX IMVI | 103.19 |
| | MALME-3M | 102.62 |
| | M14 | 101.18 |
| | MDA-MB-435 | 102.57 |
| | SK-MEL-2 | N.T. |
| | SK-MEL-28 | 107.03 |
| | SK-MEL-5 | 92.50 |
| | UACC-257 | 104.23 |
| | UACC-62 | 93.92 |
| Ovarian Cancer | IGROV1 | 81.63 |
| | OVCAR-3 | 103.33 |
| | OVCAR-4 | 98.31 |
| | OVCAR-5 | 102.33 |
| | OVCAR- 8 | 104.73 |
| | NCI/ADR-RES | 107.13 |
| | SK-OV-3 | 95.59 |
| Renal Cancer | 786-0 | 105.05 |
| | A498 | 104.44 |
| | ACHN | 95.59 |
| | CAKI-1 | 90.50 |
| | RXF 393 | 107.22 |
| | SN12C | 101.12 |
| | TK-10 | 131.97 |
| | UO-31 | 82.74 |
| Prostate Cancer | PC-3 | 93.07 |
| | DU-145 | 114.04 |
| Breast Cancer | MCF7 | 97.24 |
| | MDA-MB-231/ATCC | 93.54 |
| | HS 578T | 102.06 |
| | BT-549 | 105.34 |
| | T-47D | 95.99 |
| | MDA-MB-468 | 106.42 |
| Mean | | 100.53 |
| Delta | | 19.69 |
| Range | | 51.13 |

TABLE 8
SJ-25, SJ-26 and SJ-27 data collected from NCI

| | | SJ-25 | SJ-26 | SJ-27 |
|---|---|---|---|---|
| Compound No. | | NSC | NSC | NSC |
| NCI No. | | 763971 | 763970 | 765596 |
| Leukemia | CCRF-CEM | 71.56 | 105.78 | 89.25 |
| | HL-60 (TB) | 91.75 | 102.31 | 108.77 |
| | K-562 | 63.81 | 97.48 | 108.53 |
| | MOLT-4 | 59.49 | 95.10 | 110.33 |
| | RPMI-8226 | 62.28 | 90.60 | 90.30 |
| | SR | 61.70 | 77.02 | N.T. |
| Non-Small Cell | A549/ATCC | 76.42 | 104.88 | 99.70 |
| Lung Cancer | EKVX | 30.15 | 72.14 | N.T. |
| | HOP-62 | 91.46 | 91.26 | 96.01 |
| | HOP-92 | N.T. | N.T. | 77.75 |
| | NCI-H226 | 80.52 | 85.66 | 89.22 |
| | NCI-H23 | 66.22 | 81.56 | 93.26 |
| | NCI-H322M | 73.47 | 90.72 | 99.80 |
| | NCI-H460 | 79.22 | 83.32 | 98.27 |
| | NCI-H522 | 68.89 | 92.22 | 95.89 |
| Colon Cancer | COLO 205 | 87.39 | 98.25 | 108.91 |
| | HCC-2998 | 96.27 | 102.24 | 104.46 |
| | HCT-116 | 77.94 | 103.43 | 88.60 |
| | HCT-15 | 80.10 | 108.54 | 94.98 |
| | HT29 | 84.03 | 102.38 | 111.60 |
| | KM12 | 84.62 | 102.73 | 98.35 |
| | SW-620 | 86.99 | 95.26 | 98.89 |
| CNS Cancer | SF-268 | 96.53 | 106.64 | 113.64 |
| | SF-295 | 73.28 | 92.48 | N.T. |
| | SF-539 | 100.05 | 105.79 | 91.49 |
| | SNB-19 | 89.19 | 99.28 | 96.35 |
| | SNB-75 | 66.06 | 80.33 | 59.75 |
| | U251 | 78.04 | 96.46 | 97.67 |
| Melanoma | LOX IMVI | 68.41 | 86.91 | 116.91 |
| | MALME-3M | 107.23 | 103.37 | 107.54 |
| | M14 | 98.89 | 108.50 | 96.50 |
| | MDA-MB-435 | 95.15 | 100.35 | 100.31 |
| | SK-MEL-2 | 95.85 | 117.12 | 109.44 |
| | SK-MEL-28 | 86.90 | 100.02 | 106.24 |
| | SK-MEL-5 | 68.56 | 84.87 | 98.51 |
| | UACC-257 | 106.91 | 106.31 | 98.20 |
| | UACC-62 | 84.47 | 95.17 | 74.08 |
| Ovarian Cancer | IGROV1 | 69.73 | 91.28 | 96.92 |
| | OVCAR-3 | 89.42 | 117.80 | 111.89 |
| | OVCAR-4 | 73.19 | 80.74 | N.T. |
| | OVCAR-5 | 112.54 | 126.77 | 96.81 |
| | OVCAR-8 | 90.21 | 107.63 | 98.75 |
| | NCPADR-RES | 73.58 | 97.00 | 94.29 |
| | SK-OV-3 | N.T. | N.T. | 119.79 |
| Renal Cancer | 786-0 | 100.58 | 114.55 | 103.51 |
| | A498 | N.T. | N.T. | 93.51 |
| | ACHN | 88.12 | 104.75 | 90.83 |
| | CAKI-1 | 41.86 | 71.62 | 90.65 |
| | RXF 393 | 79.39 | 99.93 | 114.49 |
| | SN12C | 84.58 | 96.69 | 89.48 |
| | TK-10 | 99.82 | 126.63 | N.T. |
| | UO-31 | 56.23 | 70.50 | 55.69 |
| Prostate Cancer | PC-3 | 58.35 | 93.44 | 74.44 |
| | DU-145 | 92.27 | 111.80 | 121.06 |
| Breast Cancer | MCF7 | 67.82 | 68.09 | 71.98 |
| | MDA-MB-231/ | 72.56 | 86.98 | 74.61 |
| | ATCC | | | |
| | HS 578T | 58.28 | 105.98 | 92.74 |
| | BT-549 | 92.88 | 120.08 | 92.95 |
| | T-47D | 63.89 | 67.23 | 78.97 |
| | MDA-MB-486 | 77.36 | 80.08 | 99.51 |
| Mean | | 79.52 | 96.60 | 96.23 |
| delta | | 49.37 | 29.37 | 40.54 |
| Range | | 82.39 | 59.54 | 65.37 |

TABLE 9
The GI₅₀, TGI and LC₅₀ data of TC-XCI, SJ-3
and SJ-10 collected from NCI

| | (TC-XC1) (NSC763972) | | | | | SJ-3 (NSC772864) | | | | | SJ-10 (NSC772862) | | | | |
|---|---|---|---|---|---|---|---|---|---|---|---|---|---|---|---|
| Panel/ | | | | TGI | LC₅₀ | | | | TGI | LC₅₀ | | | | TGI | LC₅₀ |
| Cell | | | | Sub- | Se- | | | | Sub- | Se- | | | | Sub- | Se- |
| line | | | | panel | lectivity | | | | panel | lectivity | | | | panel | lectivity |
| (μM) | GI₅₀ | | | MID^{b} | ratio | GI₅₀ | | | MID^{b} | ratio | GI₅₀ | | | MID^{b} | ratio |
| Leukemia | | | | | | | | | | | | | | | |
| CCRF-CEM | 1.42 | 1.114 | 8.063 | 15.5 | >100 | 8.24 | 9.696 | 1.475 | 3.50 | 7.80 | 6.32 | 11.000 | 0.954 | >100 | >100 |
| HL-60(TB) | 1.06 | | | 4.28 | >100 | N.T. | | | N.T. | N.T. | 36.9 | | | >100 | >100 |
| K-562 | 0.315 | | | >100 | >100 | 8.75 | | | 3.61 | 7.46 | 7.80 | | | >100 | >100 |
| MOLT-4 | 1.97 | | | 15.3 | >100 | 6.24 | | | 3.43 | 7.91 | 4.41 | | | >100 | >100 |
| RPMI-8226 | 0.549 | | | >100 | >100 | 20.20 | | | 4.31 | 9.73 | 7.48 | | | >100 | >100 |
| SR | 1.37 | | | 43.4 | >100 | 5.05 | | | 4.06 | 11.60 | 3.09 | | | >100 | >100 |
| Non-Small Cell | | | | | | | | | | | | | | | |
| Lung Cancer | | | | | | | | | | | | | | | |
| A549/ATCC | 2.95 | 5.840 | 1.538 | >100 | >100 | 15.20 | 11.819 | 1.210 | 3.51 | 6.72 | 4.87 | 6.477 | 1.621 | >100 | >100 |
| EKVX | N.T. | | | N.T. | N.T. | N.T. | | | N.T. | N.T. | N.T. | | | N.T. | N.T. |
| HOP-62 | 3.84 | | | >100 | >100 | 4.51 | | | 2.83 | 5.74 | 12.3 | | | >100 | >100 |
| HOP-92 | 1.21 | | | 13.4 | >100 | 3.08 | | | 5.80 | 25.10 | N.T. | | | N.T. | N.T. |
| NCI-H226 | 11.6 | | | >100 | >100 | 1.88 | | | — | — | 7.77 | | | >100 | >100 |
| NCI-H23 | 3.44 | | | 63.6 | >100 | 2.63 | | | 4.00 | 8.35 | 6.65 | | | >100 | >100 |
| NCI-H322M | 16 | | | 92.3 | >100 | 34.50 | | | 13.10 | 36.20 | 5.62 | | | >100 | >100 |
| NCI-460 | 7.44 | | | 86.3 | >100 | 5.15 | | | 3.20 | 6.74 | 4.15 | | | >100 | >100 |
| NCI-H522 | 0.238 | | | 0.644 | 3.57 | 27.60 | | | 3.80 | 7.23 | 3.98 | | | >100 | >100 |
| Colon Cancer | | | | | | | | | | | | | | | |
| COLO 205 | 3.77 | 9.560 | 0.939 | 15.8 | 39.8 | 2.41 | 18.400 | 0.778 | — | — | 12.2 | 9.763 | 1.075 | >100 | >100 |
| HCC-2998 | 11.0 | | | >100 | >100 | >100 | | | 4.08 | 7.94 | 30.9 | | | >100 | >100 |
| HCT-116 | 0.807 | | | >100 | >100 | 6.19 | | | 3.19 | 6.06 | 5.42 | | | >100 | >100 |
| HCT-15 | 0.716 | | | >100 | >100 | 4.37 | | | 3.51 | 7.50 | 4.34 | | | >100 | >100 |
| HT29 | 39.0 | | | >100 | >100 | 4.23 | | | 3.16 | 6.28 | 7.84 | | | >100 | >100 |
| KM12 | 4.62 | | | >100 | >100 | 6.48 | | | 3.11 | 5.96 | 2.50 | | | >100 | >100 |
| SW-620 | 7.01 | | | >100 | >100 | 5.12 | | | 3.06 | 6.67 | 5.14 | | | >100 | >100 |
| CNS Cancer | | | | | | | | | | | | | | | |
| SF-268 | 4.28 | 14.364 | 0.625 | >100 | >100 | 17.70 | 14.875 | 0.962 | 3.27 | 6.52 | 8.71 | 8.053 | 1.304 | >100 | >100 |
| SF-295 | 10.8 | | | 87.9 | >100 | 17.80 | | | 4.60 | 9.85 | 7.79 | | | >100 | >100 |
| SF-539 | 23.1 | | | 64.2 | >100 | 20.90 | | | 3.15 | 5.86 | 14.2 | | | >100 | >100 |
| SNB-19 | 30.9 | | | >100 | >100 | 18.20 | | | 3.42 | — | 8.27 | | | >100 | >100 |
| SNB-75 | N.T. | | | N.T. | >100 | 3.25 | | | 2.35 | 4.86 | 4.53 | | | >100 | >100 |
| U251 | 2.74 | | | >100 | >100 | 11.40 | | | 3.06 | 5.88 | 4.82 | | | >100 | >100 |
| Melanoma | | | | | | | | | | | | | | | |
| LOX IMV1 | 0.64 | 10.637 | 0.844 | 57.00 | >100 | 6.04 | 15.654 | 0.914 | 3.04 | 6.02 | 4.74 | 19.894 | 0.528 | >100 | >100 |
| MALME-3M | 8.32 | | | 42.20 | >100 | 17.30 | | | 3.83 | 7.46 | 8.87 | | | >100 | >100 |
| M14 | 3.75 | | | >100 | >100 | 16.50 | | | 5.48 | 19.30 | 7.49 | | | >100 | >100 |
| MDA-MB-435 | 3.97 | | | >100 | >100 | 20.20 | | | 3.86 | 7.62 | 7.01 | | | >100 | >100 |
| SK-MEL-2 | 17.10 | | | 50.20 | >100 | 37.70 | | | 4.73 | 10.40 | 7.36 | | | >100 | >100 |
| SK-MEL-28 | 33.00 | | | >100 | >100 | 6.00 | | | 3.21 | 5.83 | >100 | | | >100 | >100 |
| SK-MEL-5 | 7.36 | | | 22.50 | 56.5 | 1.15 | | | 2.61 | — | 4.10 | | | >100 | >100 |
| UACC-257 | 6.39 | | | 65.10 | >100 | 21.20 | | | 3.39 | 6.37 | 35.1 | | | >100 | >100 |
| UACC-62 | 15.20 | | | 88.40 | >100 | 14.80 | | | 3.31 | 6.68 | 4.38 | | | >100 | >100 |
| Ovarian Cancer | | | | | | | | | | | | | | | |
| IGROV1 | 14.3 | 13.649 | 0.658 | >100 | >100 | 11.20 | 17.448 | 0.820 | 3.62 | 7.82 | 5.94 | 13.591 | 0.772 | >100 | >100 |
| OVCAR-3 | 4.46 | | | 95.1 | >100 | N.T. | | | N.T. | N.T. | 12.8 | | | >100 | >100 |
| OVCAR-4 | 8.83 | | | 21.9 | 49.30 | 2.99 | | | 2.79 | 5.68 | 6.39 | | | >100 | >100 |
| OVCAR-5 | 16.1 | | | 66.1 | >100 | 21.50 | | | 3.32 | 6.52 | 34.5 | | | >100 | >100 |
| OVCAR-8 | 1.49 | | | >100 | >100 | 13.80 | | | 3.63 | 7.76 | 8.66 | | | >100 | >100 |
| NCI/ADR-RES | 2.26 | | | 25.2 | >100 | 19.30 | | | 3.67 | 7.41 | 22.5 | | | >100 | >100 |
| SK-OV-3 | 48.1 | | | >100 | >100 | 35.90 | | | 5.57 | 17.20 | 4.35 | | | >100 | >100 |
| Renal Cancer | | | | | | | | | | | | | | | |
| 786-O | 23.4 | 10.988 | 0.817 | >100 | >100 | 26.80 | 12.733 | 1.124 | 3.29 | 6.01 | 23.5 | 6.370 | 1.648 | >100 | >100 |
| A498 | 6.75 | | | 71.7 | >100 | 8.76 | | | 1.78 | 50.80 | 2.63 | | | >100 | >100 |
| ACHN | 4.2 | | | >100 | >100 | 6.44 | | | 3.04 | 5.89 | 3.14 | | | >100 | >100 |
| CAKI-1 | 7.92 | | | >100 | >100 | 14.80 | | | 3.26 | 6.80 | 2.52 | | | >100 | >100 |
| RXF 393 | 17.9 | | | 63.6 | >100 | N.T. | | | N.T. | N.T. | 4.55 | | | >100 | >100 |
| SN12C | 3.24 | | | >100 | >100 | 4.12 | | | 2.87 | 6.16 | 5.42 | | | >100 | >100 |
| TK-10 | 22.7 | | | 68.9 | >100 | 24.60 | | | 3.70 | 6.38 | N.T. | | | N.T. | N.T. |
| UO-31 | 1.79 | | | 35.3 | >100 | 3.61 | | | 2.88 | 6.48 | 2.83 | | | >100 | >100 |
| Prostate Cancer | | | | | | | | | | | | | | | |
| PC-3 | 0.606 | 2.968 | 3.026 | >100 | >100 | 12.50 | 15.650 | 0.914 | 3.82 | 8.98 | 5.93 | 5.360 | 1.959 | >100 | >100 |
| DU-145 | 5.33 | | | >100 | >100 | 18.80 | | | 2.94 | 5.43 | 4.79 | | | >100 | >100 |
| Breast Cancer | | | | | | | | | | | | | | | |
| MCF7 | 7.73 | 7.280 | 1.234 | >100 | >100 | 0.44 | 12.345 | 1.159 | 2.60 | 5.54 | 5.42 | 6.813 | 1.541 | >100 | >100 |
| MDA-MB-231/ | 2.44 | | | 13.8 | 75.1 | 2.23 | | | 2.71 | 5.70 | 7.23 | | | >100 | >100 |
| ATCC | | | | | | | | | | | | | | | |
| HS 578T | 20.3 | | | >100 | >100 | 19.30 | | | 10.00 | 46.10 | 4.77 | | | >100 | >100 |
| BT-549 | 7.55 | | | >100 | >100 | 32.50 | | | 9.50 | 45.50 | 10.6 | | | >100 | >100 |
| T-47D | 0.84 | | | 2.86 | 8.96 | 17.80 | | | 3.56 | 7.19 | 8.44 | | | >100 | >100 |
| MDA-MB-468 | 4.82 | | | 29.4 | >100 | 1.71 | | | 3.07 | — | 4.42 | | | >100 | >100 |
| MID^{a} | 8.982 | | | | | 14.306 | | | | | 10.498 | | | | |

MID^{a}
MID^{a} = Average sensitivity of all cell line in μM.
MID^{b} = Average sensitivity of all cell line of a particular subpanel in μM.
Selectivity ratio = MID^{a}:MID^{b}
N.T. = No test

What is claimed is:

1. A compound as shown in formulation (I): 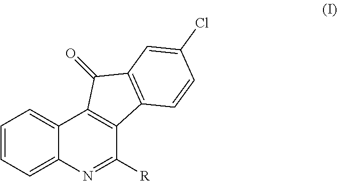
wherein the R is selected from the groups consisting of:
i) haloformyl, amino, hydroxyl and thiol groups;
ii) linear alkyl chains of N(CH₂)ₙ—H, alkyl groups with substituted side chains and alkyl side chains with a substituted amino group, NH(CH₂)ₙN(CH₂)ₙ, O(CH₂)ₙ and S(CH₂)ₙ—OH, wherein 1≦n≦10;
iii) nitrogen-containing cycloalkyl groups and heterocyclic compounds of C₃₋₁₂ which contain 1 to 3 heteroatoms selected from O, S and N, wherein the ortho-, para- and meta-position can be further selected independently from one of the groups comprising of: (CH₂)ₙ alkyl groups, (CH₂)ₙC₃₋₁₂ cycloalkyl groups, (CH₂)ₙC₃₋₁₂ nitrogen-containing cycloalkyl groups, (CH₂)ₙ benzene rings and (CH₂)ₙCOC₃₋₁₂ nitrogen-containing cycloalkyl groups, wherein 0≦n≦10;
wherein the nitrogen-containing cycloalkyl groups or the benzene rings can be further substituted by one or more substitution groups selected from the following groups comprising alkyl groups containing C₁₋₁₂, amino groups, nitro groups, hydroxyl groups, cyano groups, halogen groups, un-substituted or halogen group substituted C₁₋₅ alkyl groups, un-substituted or halogen group substituted alkoxy groups, and their pharmaceutically acceptable salts, stereoisomers and enantimoers.

2. The compound according to claim 1, wherein the functional group ii) is selected from the group consisting of methyl amine, dimethyl amine, 2-(diethylamino) ethyl amine, and 2-hydroxyethylthio.

3. The compound according to claim 1, wherein the functional group iii) is selected from the groups consisting of pyrrolidin-1-yl, piperidin-1-yl, 4-methyl-piperazin-1-yl, azepan-1-yl, morpholino, thiomorpholino, piperazin-1-yl, 2-methyl-piperazin-1-yl, 4-methyl-piperazin-1-yl, 4-ethyl-piperazin-1-yl, 4-cyclopentyl-piperazin-1-yl, 4-(piperidin-1-yl) piperidin-1-yl, 4-phenyl-piperazin-1-yl, 4-benzyl-piperazin-1-yl, 4-(2-fluorophenyl)piperazine-1-yl, 4-(2-methoxyphenyl)piperazin-1-yl, 4-(3-methoxyphenyl) piperazin-1-yl, 4-(1-methyl-piperidin-4-yl)piperazin-1-yl, 4-(1,4-dioxo-8-aza-spiro[4,5]dec-8-yl, 4-((piperazin-1-yl) (piperidin-1-yl) methanone), 4-(3-(piperidin-4-yl) propyl) piperidin-1-yl, hydroxyl, and methoxyl.

4. The compound according to claim 1, wherein the compound is selected from the groups consisting of:
9-Chloro-6-(methylamino)-11H-indeno[1,2-c]quinolin-11-one,
9-chloro-6-(dimethylamino)-11H-indeno[1,2-c]quinolin-11-one,
9-chloro-6-(pyrrolidin-1-yl)-11H-indeno[1,2-c]quinolin-11-one,
9-chloro-6-(piperidin-1-yl)-11H-indeno[1,2-c]quinolin-11-one,
9-chloro-6-(4-methyl-piperazin-1-yl)-11H-indeno[1,2-c]quinolin-11-one,
6-(azepan-1-yl)-9-chloro-11H-indeno[1,2-c]quinolin-11-one,
9-chloro-6-morpholino-11H-indeno[1,2-c]quinolin-11-one,
9-chloro-6-thiomorpholino-11H-indeno[1,2-c]quinolin-11-one,
9-chloro-6-(piperazin-1-yl)-11H-indeno[1,2-c]quinolin-11-one,
9-chloro-6-(2-methyl-piperazin-1-yl)-11H-indeno[1,2-c]quinolin-11-one,
9-chloro-6-(4-methyl-piperazin-1-yl)-11H-indeno[1,2-c]quinolin-11-one,
9-chloro-6-(4-ethyl-piperazin-1-yl)-11H-indeno[1,2-c]quinolin-11-one,
9-chloro-6-(4-cyclopentyl-piperazin-1-yl)-11H-indeno[1,2-c]quinolin-11-one,
9-chloro-6-(4-(piperidin-1-yl) piperidin-1-yl)-11H-indeno[1,2-c]quinolin-11-one,
9-chloro-6-(4-phenyl-piperazin-1-yl)-11H-indeno[1,2-c]quinolin-11-one,
6-(4-benzyl-piperazin-1-yl)-11H-indeno[1,2-c]quinolin-11-one,
9-chloro-6-(4-(2-fluorophenyl)piperazin-1-yl)-11H-indeno[1,2-c]quinolin-11-one,
9-chloro-6-(4-(2-methoxyphenyl)piperazin-1-yl)-11H-indeno[1,2-c]quinolin-11-one,
9-chloro-6-(4-(3-methoxyphenyl)piperazin-1-yl)-11H-indeno[1,2-c]quinolin-11-one,
9-chloro-6-(4-(1-methyl-piperidin-4-yl)piperazin-1-yl)-11H-indeno[1,2-c]quinolin-11-one,
9-chloro-6-(4-(1,4-dioxo-8-aza-spiro[4,5]dec-8-yl)-11H-indeno[1,2-c]quinolin-11-one,
9-chloro-6-(4-((piperazin-1-yl) (piperidin-1-yl) methanone))-11H-indeno[1,2-c]quinolin-11-one, and
9-chloro-6-(4-(3-(piperidin-4-yl) propyl) piperidin-1-yl)-11H-indeno[1,2-c]quinolin-11-one,
6-(2-Hydroxyethylthio)-9-chloro-11H-indeno[1,2-c]quinolin-11-one,
6-hydroxy-9-chloro-11H-indeno[1,2-c]quinolin-11-one,
6-methoxy-9-chloro-11H-indeno[1,2-c]quinolin-11-one,
and their salts.

5. A pharmaceutical composition comprising the effective dosage compound according to claim 1 and at least one pharmaceutically acceptable vehicle, diluent or excipient.

6. A method for inhibiting Topoisomerase I activity which comprises administration an effective amount of the compound according to claim 1 at effective doses.

7. A method for the treatment of cancers which comprises administrating an effective amount of the compound according to claim 1.

8. The method according to claim 7, wherein the cancers are selected from the groups consisting of leukemia, non-small cell lung cancer, colorectal cancer, central nervous system (CNS) cancer, melanoma, ovarian cancer, renal cancer, prostate cancer and breast cancer.

9. A method for preparation of indeno[1,2-c]quinolin-11-one derivatives, wherein the method comprising:
(1) mix isatin, 2-(4-chlorophenyl) acetic acid and sodium acetate at 200° C. for 3 hours, allow cooling before adding acetic acid and collect the precipitate following extraction and filtration; wash the precipitate with acetic acid, water and n-hexane to give an intermediate product and add the intermediate product to phosphoryl trichloride and reflux at 150° C. for 48 hours; upon completion, allow cooling to room temperature followed by addition of 0° C. ice water; collect the resulting precipitate after extraction and filtration and place in 10% aqueous sodium bicarbonate solution for 1 hour with vigorous stirring; the crude product was recrystallized from dichloromethane after washing with water to give 6,9-dichloro-11H-indeno[1,2-c]quinoline-11-one (TC-XCl-1);
(2) dissolve 6,9-dichloro-11H-indeno[1,2-c]quinoline-11-one obtained from step 1 in N,N-dimethylformamide and add methylamine or N1,N1-diethylethane-1,2-diamine followed by addition of N,N-diisopropylethylamine to catalyze the reaction at 150° C. for 4 hours; pour the resulting mixture into ice water and incubate for 10 to 20 minutes to give the precipitate which was then recrystallized from ethanol to produce the compounds 9-chloro-6-(dimethylamino)-11H-indeno[1,2-c]quinolin-11-one (SJ-1) and 6-(2-(diethylamino)ethylamine yl)-11H-indeno[1,2-c]quinolin-11-one (SJ-3);
(3) dissolve the 6,9-dichloro-11H-indeno[1,2-c]quinoline-11-one obtained from step (1) in dimethyl formamide, add secondary amines followed by addition of pyridine to catalyze the reaction at 150° C. for 4 hours; pour the resulting mixture into ice water and incubate for 10 to 20 minutes to give the precipitate which was then recrystallized from ethanol to produce the compounds which are selected from the groups consisting of
9-chloro-6-(dimethylamino)-11H-indeno[1,2-c]quinolin-11-one,
9-chloro-6-(pyrrolidin-1-yl)-11H-indeno[1,2-c]quinolin-11-one,
9-chloro-6-(piperidin-1-yl)-11H-indeno[1,2-c]quinolin-11-one,
9-chloro-6-(4-methyl-piperazin-1-yl)-11H-indeno[1,2-c]quinolin-11-one,
6-(azepan-1-yl)-9-chloro-11H-indeno[1,2-c]quinolin-11-one,
9-chloro-6-morpholino-11H-indeno[1,2-c]quinolin-11-one,
9-chloro-6-thiomorpholino-11H-indeno[1,2-c]quinolin-11-one,
9-chloro-6-(piperazin-1-yl)-11H-indeno[1,2-c]quinolin-11-one,
9-chloro-6-(2-methyl-piperazin-1-yl)-11H-indeno[1,2-c]quinolin-11-one,
9-chloro-6-(4-methyl-piperazin-1-yl)-11H-indeno[1,2-c]quinolin-11-one,
9-chloro-6-(4-ethyl-piperazin-1-yl)-11H-indeno[1,2-c]quinolin-11-one,
9-chloro-6-(4-cyclopentyl-piperazin-1-yl)-11H-indeno[1,2-c]quinolin-11-one,
9-chloro-6-(4-(piperidin-1-yl) piperidin-1-yl)-11H-indeno[1,2-c]quinolin-11-one,
9-chloro-6-(4-phenyl-piperazin-1-yl)-11H-indeno[1,2-c]quinolin-11-one,
6-(4-benzyl-piperazin-1-yl)-11H-indeno[1,2-c]quinolin-11-one,
9-chloro-6-(4-(2-fluorophenyl)piperazin-1-yl)-11H-indeno[1,2-c]quinolin-11-one,
9-chloro-6-(4-(2-methoxyphenyl)piperazin-1-yl)-11H-indeno[1,2-c]quinolin-11-one,
9-chloro-6-(4-(3-methoxyphenyl)piperazin-1-yl)-11H-indeno[1,2-c]quinolin-11-one,
9-chloro-6-(4-(1-methyl-piperidin-4-yl)piperazin-1-yl)-11H-indeno[1,2-c]quinolin-11-one,
9-chloro-6-(4-(1,4-dioxo-8-aza-spiro[4,5]dec-8-yl)-11H-indeno[1,2-c]quinolin-11-one,
9-chloro-6-(4-((piperazin-1-yl) (piperidin-1-yl) methanone))-11H-indeno[1,2-c]quinolin-11-one, and
9-chloro-6-(4-(3-(piperidin-4-yl) propyl) piperidin-1-yl)-11H-indeno[1,2-c]quinolin-11-one;
(4) dissolve the 6,9-dichloro-11H-indeno[1,2-c]quinoline-11-one obtained from step (1) in dimethyl formamide, add 2-mercaptoethanol followed by addition of potassium carbonate to catalyze the reaction at 150° C. for 4 hours; pour the resulting mixture into ice water and incubate for 10 to 20 minutes to give the precipitate which was then recrystallized from ethanol to produce the compound 6-(2-hydroxy-ethylthio)-9-chloro-11H-indeno[1,2-c]quinolin-11-one;
(5) dissolve 6,9-dichloro-11H-indeno[1,2-c]quinoline-11-one obtained from step 1 in dimethyl formamide and then add conc. hydrochloric acid at 150° C. for 24 hours. At the end of reaction, pour the mixture into ice water and incubate for 10 to 20 minutes. Finally, recrystallize the precipitate with ethanol to give the compound 6-hydroxy-9-chloro-11H-indeno[1,2-c]quinolin-11-one;
(6) dissolve 6,9-dichloro-11H-indeno[1,2-c]quinoline-11-one obtained from step 1 in methanol then add sodium methoxide at 90° C. for 10 hours. At the end of reaction, cool the mixture and recrystallize the precipitate with ethanol to give the compound 6-methoxy-9-chloro-11H-indeno[1,2-c]quinolin-11-one.
